SEQUENCE LISTING (8 sequences)

SEQ ID NO: 1 - DNA, 10 bp, synthetic construct
  misc_feature 1..10: note = Description of Artificial Sequence: Synthetic oligonucleotide
  misc_feature 1..10: note = Based on human, animal, plant or microorganism
  source 1..10: mol_type = other DNA
acaacactgt

SEQ ID NO: 2 - DNA, 11 bp, synthetic construct
  misc_feature 1..11: note = Description of Artificial Sequence: Synthetic oligonucleotide
  misc_feature 1..11: note = Based on human, animal, plant or microorganism
  source 1..11: mol_type = other DNA
cacaacactgt

SEQ ID NO: 3 - DNA, 12 bp, synthetic construct
  misc_feature 1..12: note = Description of Artificial Sequence: Synthetic oligonucleotide
  misc_feature 1..12: note = Based on human, animal, plant or microorganism
  source 1..12: mol_type = other DNA
ccacaacactgt

SEQ ID NO: 4 - DNA, 11 bp, synthetic construct
  misc_feature 1..11: note = Description of Artificial Sequence: Synthetic oligonucleotide
  misc_feature 1..11: note = Based on human, animal, plant or microorganism
  source 1..11: mol_type = other DNA
ccacaacctgt

SEQ ID NO: 5 - DNA, 10 bp, synthetic construct
  misc_feature 1..10: note = Description of Artificial Sequence: Synthetic oligonucleotide
  misc_feature 1..10: note = Based on human, animal, plant or microorganism
  source 1..10: mol_type = other DNA
ccacaactgt

SEQ ID NO: 6 - DNA, 18 bp, synthetic construct
  misc_feature 1..18: note = Description of Artificial Sequence: Synthetic oligonucleotide
  source 1..18: mol_type = other DNA
ccacaacactgtatgatt

SEQ ID NO: 7 - DNA, 16 bp, synthetic construct
  misc_feature 1..16: note = Description of Artificial Sequence: Synthetic oligonucleotide
  source 1..16: mol_type = other DNA
acaacactgtatgatt

SEQ ID NO: 8 - DNA, 10 bp, synthetic construct
  misc_feature 1..10: note = Description of Artificial Sequence: Synthetic oligonucleotide
  source 1..10: mol_type = other DNA
ctgtatgatt

(19) United States
(12) Patent Application Publication
(10) Pub. No.: US 2026/0204347 A1
(43) Pub. Date: Jul. 16, 2026

(54) METHODS FOR DNA LIBRARY GENERATION TO FACILITATE THE DETECTION AND REPORTING OF LOW FREQUENCY VARIANTS

(71) Applicant: Sophia Genetics S.A., Rolle (CH)

(72) Inventors: Morgane MACHERET, Saint-Sulpice (CH); Christian POZZORINI, Lausanne (CH); Adrian WILLIG, Geneva (CH); Jonathan BIELER, Renens (CH); Zhenyu XU, Nyon (CH); Lin SONG, Renens (CH); Frédéric Michaud, Lausanne (CH)

(73) Assignee: Sophia Genetics S.A., Rolle (CH)

(21) Appl. No.: 19/357,459

(22) Filed: Oct. 14, 2025

Related U.S. Application Data
(63) Continuation-in-part of application No. 17/438,461, filed on Sep. 21, 2020.

(30) Foreign Application Priority Data
Sep. 20, 2019 (EP) 19198542.3

(51) Int. Cl. G16B 20/20; C12Q 1/6806; C12Q 1/686; C12Q 1/6869; G16B 30/10; G16H 15/00; G16H 50/20; G16H 50/70

(52) U.S. Cl. CPC G16B 20/20; C12Q 1/6806; C12Q 1/686; C12Q 1/6869; G16B 30/10; G16H 15/00; G16H 50/20; G16H 50/70

(57) ABSTRACT

Methods and products are disclosed for adapting fragmented nucleic acids for next generation sequencing, including providing numerical codes based on variable adapter lengths for the strand and the fragment, respectively, of the fragmented nucleic acids. The methods and products allow for the amplification of the fragmented nucleic acids when there is a low yield of isolated fragmented nucleic acids and also for efficient and reliable detection of low-frequency mutations including in subpopulations of cells within a subject.

### CROSS REFERENCE TO RELATED APPLICATIONS

This application is a continuation in part application to U.S. patent application Ser. No. 17/438,461 (filed Sep. 12, 2021), which claims the benefit of priority to International PCT Application No.: PCT/EP2020/076246 (filed Sep. 21, 2020), which claims benefit of European Patent Application No.: 19198542.3, (filed Sep. 20, 2019), which is incorporated by reference herein in its entirety.

### REFERENCE TO ELECTRONIC SEQUENCE LISTING

This application contains a Sequence Listing which has been submitted electronically in .XML format and is hereby incorporated by reference in its entirety. Said .XML copy, created on Feb. 17, 2026, is named 1200017PCTUSCON_SEQLIST_ST26.xml and is 13,617 bytes in size. The sequence listing contained in this .XML file is part of the specification and is hereby incorporated by reference herein in its entirety.

### BACKGROUND OF THE INVENTION

Fields, such as cancer therapeutics, forensics, paleo-genomics, evolution and toxicology, require high-accuracy sequencing and detection of low incidence mutations. Such mutations might even be present in less than 1% of the cells, such as with cancer. When analyzing cell-free deoxyribonucleic acid (DNA) fragments from a plasma or blood sample, the ratio of DNA fragments from tumor cells may even be as low as 0.01% of the total cell-free DNA. This low incidence-genetic diversity is difficult to assess with conventional next generation sequencing due to a high background error rate not only in the sequencing itself, but in the amplification of the genomic DNA prior to sequencing. Circulating tumor DNA fragments may be fragmented to an average length of 140 to 180 bp (base pairs) and present in only a few thousands amplifiable copies per millimeter of blood. DNA polymerases can introduce mis-insertions at a frequency of 10⁻⁴ to 10⁻⁶.

When these mis-insertions occur early in the generation of the DNA library, such as during first strand synthesis, they can become indistinguishable from low-frequency mutations. Moreover, high throughput sequencing systems, also known as Next-Generation-Sequencing (NGS) systems typically produce errors at a per-base rate of 10⁻² to 10⁻³, making certain true variants undetectable when the corresponding mutations occur at a similar or lower frequency.

For example, single-cell sequencing, single-stranded molecular barcoding, and circle sequencing may involve sequencing DNA derived from a single strand of DNA. During the first round of amplification, DNA polymerase may propagate errors to the daughter molecules. In single-cell sequencing, random primers may be used with a DNA polymerase with helicase activity to displace one of the two strands. But the combination of random primers and strand displacement can result in random priming of the newly copied strand and thus, the generation of copies of copies. In the process, any initial misincorporation error will be passed to the copies of copies. As all the genetic information was derived from a single cell, it is impossible to tell whether the sequencing reads represent an error from the original single-strand synthesis or a genetic variant.

CircSeq and single-stranded barcoding may also introduce mis-insertions during first round synthesis, an error which may then be propagated to daughter molecules and erroneously scored as a mutation. The same mis-insertion error post-isolation is unlikely to occur in the same DNA sequence from other cells or sub-clonal populations. The original error therefore, could not necessarily be identified, accounted for, and/or corrected via post-hoc analysis, instead resulting in errors that may appear to be a sub-clonal mutations.

In “Enhancing the accuracy of next-generation sequencing for detecting rare and subclonal mutations”, Nature Reviews-Genetics, Vol. 18, pp. 269-285, May 2018 Salk et al. review three main error correction strategies to better characterize low frequency variants with NGS technologies: 1) computational strategies based on filtering low confidence data and/or applying predefined statistical models of the sequencing error profiles 2) experimental strategies to reduce the errors caused by the pre-sequencing DNA library preparation and 3) molecular consensus sequencing, which applies a posteriori detection and correction of errors in the sequencing data reads themselves. The latter methods rely upon a unique tagging with a molecular barcode (also known as molecular tag, Unique Molecular Identifier UMI, or Single Molecular Identifier SMI) of each of the DNA fragments prior to amplification and sequencing, so that it is possible to group sequencing reads in families of reads associated with a specific tag. This facilitates the explicit detection and correction of errors introduced after the tagging, as it is unlikely that the very same error systematically repeats over all amplified and sequenced amplicon copies of the uniquely tagged parent DNA fragment. Salk et al. distinguish between exogenous molecular barcodes as random or semi-random sequences which are artificially (physically) incorporated into either the PCR primers or the sequencing adaptors on the one hand, and endogenous molecular barcodes which may be identified as naturally (virtually) occurring fragmentation points (also known as shear points) at the ends of DNA molecules when preparing the DNA library using ligation. The use of UMIs to improve differential coverage, label molecules for the purpose of counting and tracking molecules after sequencing is further discussed in Kivioja et al., Nat Methods. 2011 Nov. 20; 9(1):72-4, Fu et al., Proc Natl Acad Sci USA. 2011 May 31; 108(22):9026-31, Schmitt et al., Proc Natl Acad Sci USA. 2012 Sep. 4; 109(36):14508-13, and Kinde et al., Proc Natl Acad Sci USA. 2011 Jun. 7; 108(23):9530-5.

Three main families of molecular consensus sequencing have been developed so far: 1) Single-Strand Consensus Sequencing, such as for instance the SafeSeqS, smMIP and CiqSed methods, which independently tags either one or both of the parent DNA fragment strands (thus with the limitation that it is not possible to use the strand information to group amplicon reads issued from complementary strands in the downstream consensus error detection and correction steps); 2) Two-Strand Consensus Sequencing, such as for instance the Ultrasensitive Deep Sequencing method or the CypherSeq method which tag both strands of the parent DNA fragment with the same molecular identifier so that the associated reads can be grouped into the same consensus sequence after sequencing; and 3) duplex sequencing, which introduces randomized duplex tags onto both ends of the original double-stranded DNA fragment in a complementary fashion. These molecular identifier sequences may be encoded into adaptors that are ligated to each end of a double-stranded DNA so that each end of the double-stranded DNA receives a different molecular identifier sequence. If an error is introduced by DNA polymerase into one of the two strands of DNA during first strand-synthesis or any subsequent synthesis/amplification step, the other strand provides a basis of comparison by, for example, reference to a set of single-stranded consensus sequences. Once all the single strand consensus sequences are read during sequencing, the molecular identifier sequence on each end of each strand of the original DNA fragment can be matched during alignment.

To detect post-isolation errors which occur during synthesis steps subsequent to the first-synthesis step, each strand can be aligned with its same-strand sisters, by associating the sequencing reads sharing the same start and/or end positions during alignment of the single-strand consensus sequences using the molecular identifier sequence. Any differences in the read sequence can be attributed to mis-insertions during a synthesis step subsequent to the first synthesis step. To detect post-isolation errors which occur during the first synthesis step, each strand can be aligned with its opposite-strand partner during alignment of the duplex consensus sequences (again, using the molecular identifier sequences). Any differences in the read sequences observed by such a comparison may be attributed to mis-insertions during the first synthesis step. If a particular difference is found in both partner strands of the DNA with the same molecular identifier sequence at both ends of the DNA, then the particular difference may be attributed to a mutation or polymorphism existing in the DNA as extracted from the cell. Low incidence mutations in a subset of cells can be identified during the alignment of the total sequence readout by identifying strands with substantially similar sequences but having different molecular identifier sequences.

In “Error-correcting DNA barcodes for high-throughput sequencing”, J. A. Hawkins et al, bioRxiv, 7 May 2018 proposes the use of up to more than 106 unique error-correcting barcodes by constructing a library of DNA adaptors designed according to improvements over information theory codes such as Hamming codes, Reed-Solomon codes or Levenshtein codes. WO2018/144159 proposes the use of a variable length of 2 to 24 nucleotides with a constant 3′ overhang to construct a library of DNA adaptors with another axis of diversity to facilitate the discrimination of the DNA sample fragment. Such methods may facilitate to a certain extent the inherent correction of substitution, insertion, and deletion errors, even when the corrupted length of the barcode is unknown, yet their specific design does not fully exploit the error correcting capability of the downstream sequencing data processing and variant calling workflows.

In “A review of somatic single nucleotide variant calling algorithms for next-generation sequencing data”, Computational and Structural Biotechnology Journal 16, pp. 15-24, February 2018, Xu reviews 46 publicly available variant callers which may be applicable to single nucleotide variant detection, including 4 variant callers which handle UMI-based sequencing data possibly with duplex and consensus sequencing. As reported by Xu, one limitation of current duplex sequencing protocols is that in practical experiments, only 20% of the UMIs can be matched to the other strand due to insufficient ligation efficiency, so variant calling has to process both singular and duplex UMIs. Moreover, UMI sequences themselves are prone to PCR errors, which may require complementary clustering strategies.

### SUMMARY OF THE INVENTION

This summary is provided to introduce a selection of concepts in a simplified form that are further described in the Detailed Description. This summary is not intended to identify key features or essential features, nor is it intended to limit the scope of the claims included herewith.

Aspects of the present disclosure relate to a method for detecting genetic variants in a sample, the method comprising the steps of:
a. preparing a nucleic acid for sequencing using a high-throughput sequencing platform,
wherein the preparing comprises a least one PCR amplification reaction,
wherein the preparing comprises, before the first PCR amplification reaction, incorporating molecular identifiers that can differ among molecules;
b. subjecting the preparation to high-throughput sequencing;
c. processing sequencing reads to remove adapter sequences and bad quality sequence;
d. aligning cleaned sequencing reads to a reference genome;
e. for each position in genomic regions of interest, assessing the likelihood of a genetic variant being present in the sample based on at least one aggregated plausibility metric that considers the distribution of signal among groups of reads with distinct molecular identifiers that overlap with the position.

Aspects of the present disclosure relate to a method wherein the molecular identifiers are provided by the start and end sites of the nucleic acid fragment compared to a reference genome.

Aspects of the present disclosure relate to a method wherein the molecular identifiers further include variable length spacers incorporated as part of adapters, wherein the variable length spacers include a variable subsequence that can differ among adapters and a constant termination sequence that is used to identify the end of the variable length spacer in the sequencing reads.

Aspects of the present disclosure relate to a method wherein the plausibility is calculated for each group of reads sharing the same molecular identifiers and then aggregated among groups of reads with distinct molecular identifiers overlapping the genomic position.

Aspects of the present disclosure relate to a method wherein the plausibility is calculated for each possible state, wherein the possible states represent any of distinct nucleotides, insertions of nucleotides compared to the reference genome, deletions of nucleotides compared to the reference genome, or groups of nucleotides.

Aspects of the present disclosure relate to a method wherein inferring the plausibility is based on at least one of the number of reads supporting a given state, the quality of the reads supporting a given state, the quality of the positions supporting a given state, or the quality of positions adjacent to the positions supporting a given state.

Aspects of the present disclosure relate to a method wherein the inferred plausibility is decreased if the level of support for the state differs among forward and reverse reads or among reads originating from each of the two strands in double-stranded starting molecules.

Aspects of the present disclosure relate to a method wherein calculating the plausibility for a given state involves comparing the support for the most highly supported and the support for the second most highly supported within each group of reads sharing the same molecular identifiers.

Aspects of the present disclosure relate to a method wherein assessing the likelihood of a genetic variant being present at a given genomic position in the sample involves calculating a weighted support metric based on at least one plausibility metric aggregated among groups of reads sharing the same molecular identifiers that overlap with the genomic position.

Aspects of the present disclosure relate to a method wherein the weighted support metric represents the plausibility summed across groups of reads sharing the same molecular identifiers that support the same state at a given position, the fraction of the aggregated plausibility for a given state out of the aggregated plausibility for all states, the plausibility summed across groups of reads sharing the same molecular identifiers for which both forward and reverse reads support the same state at a given position, or the plausibility summed across groups of reads sharing the same molecular identifiers for which reads originating from both strands in the original double-stranded nucleic acid support the same state at a given position.

Aspects of the present disclosure relate to a method wherein the aggregated plausibility includes a correction factor to penalize some states.

Aspects of the present disclosure relate to a method wherein the aggregated plausibility includes at least one correction factor based on at least one of the concordance between forward and reverse reads, the concordance between the reads originating from each of the two strands in the original molecule, or the amount of support expected at the position in the absence of a variant in the original sample.

Aspects of the present disclosure relate to a method wherein assessing the likelihood of a genetic variant being present at a given genomic position in the sample involves comparing an aggregated plausibility metric to a pre-defined threshold.

Aspects of the present disclosure relate to a method wherein assessing the likelihood of a genetic variant being present at a given genomic position in the sample involves comparing an aggregated plausibility metric to the value of the aggregated plausibility metric expected in the absence of a genetic variant.

Aspects of the present disclosure relate to a method wherein assessing the likelihood of a genetic variant being present at a given genomic position in the sample is based on multiple metrics, at least one of which is a plausibility metric aggregated across groups of reads with distinct molecular identifiers.

Aspects of the present disclosure relate to a method wherein the starting nucleic acid is DNA, in the form of genomic DNA, cell-free DNA, or complementary DNA obtained via reverse transcription of RNA of cell-free DNA.

Aspects of the present disclosure relate to a method wherein cell-free DNA and genomic DNA of a same subject are analyzed in parallel, wherein the support metric for each genetic variant are compared between the cell-free DNA and the paired genomic DNA to distinguish putatively somatic from putatively germline variants.

Aspects of the present disclosure relate to a method wherein the subject suffers to or is suspected to suffer from a cancer, and wherein the comparison of the support metric for each genetic variant between the cell-free DNA and the paired genomic DNA is used to distinguish putative germline variants, putative clonal hematopoiesis variants, and putative tumor variants.

Aspects of the present disclosure relate to a method wherein the genetic variants with a probability above a given threshold are reported together with support values to a user.

### BRIEF DESCRIPTION OF THE DRAWINGS

FIG. 1 shows the overall workflow to prepare a sequencing library from different starting materials.

FIG. 2 shows the overall workflow to prepare a single sequencing library from different starting materials from the same sample, wherein the starting materials are cfDNA and gDNA.

FIG. 3 is a schematic representation of a genomic analysis workflow comprising a tagging step with ligation adaptors for uniquely encoding the input DNA fragments into DNA-adaptor products in the laboratory process (also known as the “wet lab” process), and a pre-processing step on the resulting DNA-adaptor products sequencing reads to uniquely identify the DNA fragment source for each read in the bioinformatics workflow (also known as the “dry lab” process).

FIG. 4 is a schematic representation of an exemplary DNA-adaptor product for use in DNA library generation.

FIG. 5 shows a diversity of adaptors with a variable length spacer sequence partially truncated from a predefined constant sequence as may be used in the proposed method.

FIG. 6 illustrates examples of numerical codes as may be generated by the proposed method for the DNA-adaptor products associated with each DNA fragment.

FIGS. 7A-7B show examples of a set of spacer sequences SS. FIG. 7A shows one example of a set of spacer sequences SS formed by concatenating variable length truncated spacer subsequences derivatives Sᵢ with a termination sequence for producing the adaptors to be used with the proposed method. FIG. 7B shows a different example of a set of spacer sequences SS formed by concatenating variable length truncated spacer subsequences derivatives Sᵢ with a termination sequence for producing the adaptors to be used with the proposed method.

FIG. 8 illustrates an example of various DNA-adaptor PCR duplicates at the sequencing stage, out of which two duplicates can be traced back to the same parent DNA product thanks to their unique numerical codes as may be generated by the proposed method for the DNA-adaptor products associated with each DNA fragment.

FIG. 9 shows an example of pre-processing the raw sequencing reads to identify the source DNA fragment and to tag each read accordingly.

FIG. 10 shows the overall workflow of the analyses leading to the identification of different types of genetic variants.

FIG. 11 shows the overall workflow of the analyses leading to the identification of different types of genetic variants, wherein the starting material is paired gDNA and cfDNA from the same sample that can be used to distinction tumor variants, germline variants, and clonal hematopoiesis variants.

FIG. 12 shows an abstract representation of two different possible genomic analysis workflow steps to further identify variants out of the tagged reads according to the proposed methods.

FIG. 13 provides a high-level comparison of the probabilistic sequencing approach, as described herein (top) and the standard consensus approach (bottom).

FIG. 14 provides an illustration of an embodiment wherein the plausibility plaus^{N1} of a variant being present in a read group is inferred based on the number of reads covering position i in the read group j.

FIG. 15 provides an illustration of an embodiment wherein the plausibility plaus^{N1} of a variant being present in a read group is inferred based on the quality score of individual nucleotides within reads covering position i in the read group j.

FIG. 16 provides an illustration of plausibility calculations for five sequences of six base pairs each, with positions aligned vertically and numbered at the top. The number of states observed at position 4 are listed on the right, focusing on 1 bp haplotypes (single nucleotide, two states N), 3 bp haplotypes (three states N in this example) or 5 bp haplotypes (five states N in this example) centered on position 4. The resultant number of read and plausibility are indicated.

FIG. 17 provides one possible embodiment of the methods described herein. Three read group are illustrated on the left, with bars on the side indicating they share the same start end positions and molecular barcodes. The arrows represent forward reads (right direction) versus reverse reads (left direction). The black points represent variants compared to a reference. The method considers each position i consecutively, with one highlighted with a box. Plausibility metrics are calculated per read group. The read-group level plausibility are then used to calculate aggregated support metrics. In this example, the aggregated support metrics are combined with background noise, estimated independently from the sequencing data and from other sample, and prior knowledge, and a variant calling is used to call variants. The detected variants are then annotated with support metrics and information extracted from databases.

FIG. 18 provides an example of the method described herein for distinguishing between somatic, germline, and clonal hematopoietic variants.

FIG. 19 shows the ratio of reads assigned to the expected adaptor sequences after sequencing.

FIG. 20 shows the density distribution for a library of adaptors of various lengths produced according to the proposed methods.

FIGS. 21A-21B show IGV viewer screen shots of reads. FIG. 21A shows an IGV viewer screen shot of reads aligned and grouped without taking into account the proposed adaptor numerical code tagging information. FIG. 21B shows an IGV viewer screen shot of the same reads aligned and grouped according to the proposed adaptor numerical code tagging information to facilitate the identification of a heterogeneous SNP.

FIGS. 22A-22B show a comparison of the variant calling results and sequencing workflow. FIG. 22A compare the variant calling results obtained respectively when employing prior art adaptors or the proposed variable length adaptors. FIG. 22B compare the ROC curves of a consensus sequencing workflow and of a probabilistic sequencing workflow when employing respectively prior art adaptors or the proposed variable length adaptors.

### DETAILED DESCRIPTION OF THE INVENTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The proposed methods and systems will now be described by reference to more detailed embodiments. The proposed methods and systems may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

A method is described to detect genetic mutations present at low frequency in a sample. In some embodiments, the nucleic acid extracted from the sample is DNA. In some embodiments, the nucleic acid extracted from the sample is RNA. In some embodiments, both DNA and RNA are extracted from the sample.

In some embodiments, the sample corresponds to cfDNA or cfRNA extracted from blood plasma, and is used to detect tumor DNA/RNA located in another part of the body. In some embodiments, the sample corresponds to cfDNA or cfRNA isolated from maternal blood, and is used to detect fetal DNA/RNA. In some embodiments, the sample is DNA extracted from blastocyte or the polar body of an embryo pre-implantation and is used for pre-implantation genetic tests. In some embodiments, the sample is a tissue biopsy, and is used to detect tumor mutations, including subclonal mutations. In some embodiments, the sample is a blood sample and is used to detect mutations in blood cells. In some embodiments, paired tumor and normal samples are analyzed jointly. In such embodiments, the normal sample may correspond to genomic DNA (gDNA) extracted from white blood cells, while the tumor sample may correspond to DNA extracted from a biopsy or a liquid biopsy.

### Definitions

A “DNA sample” refers to a nucleic acid sample derived from an organism, as may be extracted for instance from a solid tumor or a fluid. The organism may be a human, an animal, a plant, fungi, or a microorganism. The nucleic acids may be found in limited quantity or low concentration, such as fetal circulating DNA (cfDNA) or circulating tumor DNA in blood or plasma. A DNA sample also applies herein to describe RNA samples that were reverse-transcribed and converted to cDNA.

A “DNA fragment” refers to a short piece of DNA resulting from the fragmentation of high molecular weight DNA. Fragmentation may have occurred naturally in the sample organism, or may have been produced artificially from a DNA fragmenting method applied to a DNA sample, for instance by mechanical shearing, sonification, enzymatic fragmentation and other methods. After fragmentation, the DNA pieces may be end repaired to ensure that each molecule possesses blunt ends. To improve ligation efficiency, an adenine may be added to each of the 3′ blunt ends of the fragmented DNA, enabling DNA fragments to be ligated to adaptors with complementary dT-overhangs.

A “DNA product” refers to an engineered piece of DNA resulting from manipulating, extending, ligating, duplicating, amplifying, copying, editing and/or cutting a DNA fragment to adapt it to a next-generation sequencing workflow.

A “DNA-adaptor product” refers to a DNA product resulting from ligating a DNA fragment with a DNA adaptor to adapt it to a next-generation sequencing workflow.

A “DNA library” refers to a collection of DNA products or DNA-adaptor products that were generated from DNA fragments adapted for compatibility with a next-generation sequencing workflow.

As used herein, the terms “cell-free RNA” and “cfRNA” interchangeably refer to RNA molecules that circulate in a subject's body and originate from one or more healthy cells and/or from one or more cancer cells. These RNA molecules are found outside cells, in bodily fluids such as blood, whole blood, plasma, serum, urine, cerebrospinal fluid, fecal, saliva, sweat, tears, pleural fluid, pericardial fluid, or peritoneal fluid of a subject, and are believed to be fragments of RNA expelled from healthy and/or cancerous cells, e.g., upon apoptosis and lysis of the cellular envelope. The cell-free RNA can be in the form of microvesicles, exosomes, apoptotic bodies, or RNA-protein complexes.

As used herein, the terms “cell-free DNA” and “cfDNA” interchangeably refer to DNA molecules that circulate in a subject's body and originate from one or more healthy cells and/or from one or more cancer cells. These DNA molecules are found outside cells, in bodily fluids such as blood, whole blood, plasma, serum, urine, cerebrospinal fluid, fecal, saliva, sweat, tears, pleural fluid, pericardial fluid, or peritoneal fluid of a subject, and are believed to be fragments of DNA expelled from healthy and/or cancerous cells, e.g., upon apoptosis and lysis of the cellular envelope. The cell-free DNA can be in the form of microvesicles, exosomes, apoptotic bodies, or DNA-protein complexes.

The term “Next Generation Sequencing” or “NGS” or “High-Throughput Sequencing” as used herein refers to a method of parallel sequencing. For instance, a nucleic acid (e.g., DNA) sample is obtained and prepared into a library (meaning a collection of nucleic acid fragments from the sample). The library may be prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. According to some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. The term “NGS read length” as used herein refers to the number of base pairs (bp) sequenced from a DNA fragment, or each end of a DNA fragment. After sequencing, the sequencing reads may be aligned onto a reference genome, enabling comparison between the sample and the reference.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp). In some embodiments, the sequence reads are of a mean, median or average length of about 1000 bp, 2000 bp, 5000 bp, 10,000 bp, or 50,000 bp or more. Nanopore® sequencing methods and associated devices provided by Oxford Nanopore Technology PLC of Oxford, UK, for example, can provide sequence reads that can vary in size from tens to hundreds to thousands of base pairs. Illumina® parallel sequencing methods and associated devices provided by Illumina Inc. of San Diego, CA, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

A “pool” refers to multiple DNA samples (for instance, 8 samples, 48 samples, 96 samples, or more) derived from the same or different organisms, as may be multiplexed into a single high-throughput sequencing analysis or a single step within a high-throughput sequencing workflow. Each sample may be identified in the pool by a unique sample barcode.

A “nucleotide sequence” or a “polynucleotide sequence” refers to any polymer or oligomer of nucleotides such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string). It may be DNA or RNA, or a combination thereof. It may be found permanently or temporarily in a single-stranded or a double-stranded shape. Unless otherwise indicated, nucleic acids sequences are written left to right in 5′ to 3′ orientation.

A “random sequence” or “partially random sequence” refers to a sequence of nucleotides which is at least in part randomly selected among all possible combinations of nucleotides for a given sequence length. The selection of the random sequence may be manual or automated.

A “constant sequence” or a “predefined sequence” refers to a fully specified, non-random, fixed sequence of nucleotides which is specifically selected among all possible combinations of nucleotides for a given sequence length. The selection of the non-random sequence may be manual or automated. The selection of the non-random sequence may be based upon certain criteria specific to the sequencing application and/or the sequencing technology, for instance to provide enhanced error robustness properties for amplification and sequencing steps.

A “primer sequence” refers to a nucleotide sequence comprising a region of complementarity to a target DNA a part or all of which is to be elongated or amplified.

The “edit distance” between two sequences of nucleotides refers to the minimum number of nucleotide substitutions, insertions or deletions that needs to be applied for one sequence to become identical to the other sequence.

“Ligation” refers to the joining of separate of single-stranded of double-stranded DNA sequences. The latter DNA molecules may be blunt ended or may have compatible overhangs to facilitate their ligation. Ligation may be produced by various methods, for instance using a ligase enzyme, performing chemical ligation, and other methods.

“Amplification” refers to a polynucleotide amplification reaction to produce multiple polynucleotide sequences replicated from one or more parent sequences. Amplification may be produced by various methods, for instance a polymerase chain reaction (PCR), a linear polymerase chain reaction, a nucleic acid sequence-based amplification, rolling circle amplification, and other methods.

An “adapter” or “adaptor” refers to a short double-stranded or partially double-stranded DNA molecule of around 10 to 100 nucleotides (base pairs) which has been designed to be ligated to a DNA fragment. An adaptor may have blunt ends, sticky ends as a 3′ or a 5′ overhang, or a combination thereof. For example, to improve ligation efficiency, an adenine may be added to each of the 3′ blunt ends of the fragmented DNA prior to adaptor ligation, and the adaptor may have a thymidine overhang on the 3′ end to base-pair with the adenine added to the 3′ end of the fragmented DNA. The adaptor may have a phosphorothioate bond before the terminal thymidine on the 3′ end to prevent an exonuclease from trimming the thymidine, thus creating a blunt end when the end of the adaptor being ligated is double-stranded. The adaptor may contain sequences needed for subsequent primer-binding for PCR amplification, for subsequent high-throughput sequencing, and/or for molecular barcoding.

A “partially double-stranded adaptor” refers to an adaptor including both a double-stranded region and a single stranded region. The double-stranded region of the adaptor may contain the ligation domain, whereas the single-stranded region may contain the primering sequences used for subsequent library amplification, barcoding and/or sequencing. The single stranded region can either be composed of two single stranded arms, a 5′ arm and a 3′ arm, as it is the case for so-called Y-shape adaptors, or the single stranded region of partially double stranded adaptor can form a hairpin or a loop, as it is the case for the so-called U-shape adaptors. The term partially double stranded adaptor refers thus to both Y-shape and U-shape adaptors or a combination thereof.

A “PCR duplicate”, as used herein, refers to a copy generated by PCR amplification from a single stranded DNA molecule belonging to a DNA-adaptor product derived from an original DNA fragment.

A “molecular tag” or “molecular barcode” or “molecular code” or “molecular identifier” refers to a molecular arrangement such as a nucleic acid sequence which is fully and uniquely specified by its string of nucleotides.

A “numerical code” or “non-molecular code” or “non-molecular identifier” refers to the measurement as one or more numerical values of an inherent property of a molecular arrangement, which is not the molecular arrangement itself. Examples of properties of a nucleic acid molecular sequence include length, size, molecular weight, molarity, polarity, elasticity, stiffness, electrical conductivity, fluorescence, reflectivity to certain excitation waves, or more generally any physical, chemical or biological property which may be experimentally measured for a molecular arrangement and/or parts of a molecular arrangement.

A “variable length code (VLC)” refers to the variable length of a nucleic acid sequence which may be measured as the number of nucleotides, the number of monomers, the number of polymers, the number of homopolymers, the number of heteropolymers, or a combination thereof.

“Read trimming” or “Read pre-processing” refers, in a bioinformatics workflow, to the filtering out, in the sequencing reads, of a set of nucleotides to extract the real DNA fragment sequence to be analyzed. The trimming may be performed to remove a set of nucleotides at the start of the read sequence string, such as for instance the nucleotides corresponding to the adaptor sequences. The trimming may remove a set of nucleotides at the end of the read sequence string, such as for instance to corresponding to the reverse complement of the adaptor sequence ligated at the other end of the molecule, or to bad quality nucleotides.

“Aligning” or “alignment” or “aligner” refers to mapping and aligning base-by-base, in a bioinformatics workflow, the pre-processed sequencing reads to a reference genome sequence, depending on the application. For instance, in a targeted enrichment application where the sequencing reads are expected to map to a specific targeted genomic region in accordance with the hybrid capture probes used in the experimental amplification process, the alignment may be specifically searched relative to the corresponding sequence, defined by genomic coordinates such as the chromosome number, the start position and the end position in a reference genome. Alternatively, the alignment may be searched across a reference genome corresponding to the whole genome of the species, which may also include sets of alternative alleles, and only reads aligning to a specific set of genomic regions may be considered for downstream analyses.

“Variant calling” or “variant caller” or “variant call” refers to identifying, in the bioinformatics workflow, actual variants in the aligned reads. Variants may include single nucleotide permutations (SNPs) also known as single nucleotide variants (SNVs), insertions or deletions (INDELs), copy number variants (CNVs), as well as large rearrangements, substitutions, duplications, translocations, and others. Preferably variant calling is robust enough to sort out the real variants from the amplification and sequencing noise artefacts.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (e.g., copy number variation “CNV”), a partial or complete change in the ploidy of the cell, a variation in the expression level of a gene, as well as in changes in the epigenetic information of a genome, such as altered DNA methylation patterns.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

“Consensus sequencing” refers, in a bioinformatics workflow, to grouping sequencing reads into families of reads issued from the same double-stranded DNA fragment and/or the same DNA fragment strand, comparing them to detect errors due to the amplification and/or sequencing steps, and correcting the errors to produce a unique, consensus sequence for the double-stranded DNA fragment or the DNA fragment strand. Variant calling is then performed by processing the resulting consensus sequences, rather than the totality of reads.

“Probabilistic sequencing” refers, in a bioinformatics workflow, to grouping sequencing reads into families of reads issued from the same double-stranded DNA fragment and/or the same DNA fragment strand and performing variant calling directly on this data, by processing the totality of reads from different families in order to compute the probability of data supporting all the possible genotypes at each genomic position to be analyzed, by comparing the data with a probabilistic model.

As used herein, the term “reverse transcription” or grammatical variations thereof, refers to the process of copying the nucleotide sequence of an RNA molecule into a DNA molecule. Reverse transcription can be done by reacting an RNA template with an RNA-dependent DNA polymerase (also known as a reverse transcriptase) under well-known conditions. A reverse transcriptase is a DNA polymerase that transcribes single-stranded RNA into single stranded DNA. Depending on the polymerase used, the reverse transcriptase can also have RNase H activity for subsequent degradation of the RNA template.

As used herein, the term “complementary DNA” or “cDNA” refers to a synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence.

The term “chromosomal rearrangement” as used herein refers to reorganizations of chromosome structure that can affect expression of more than one gene and the pattern of gene transmission. Usually, gene rearrangements are caused by a breakage in the DNA double helices at two different locations, followed by a rejoining of the broken ends to produce a new chromosomal arrangement of genes.

As used herein, the term “coverage” in reference to NGS refers to the number of reads that align to, or “cover” known reference basis. The coverage may be reported per position or as the average or median over multiple positions. The sequencing coverage level determines whether variant discovery can be made with a certain degree of confidence at particular base positions. Across multiple genomic regions, the average coverage can be computed as the read count multiplied by the read length and divided by the total length of the considered genomic regions. At a higher level of coverage, each base is covered by a greater number of aligned sequence reads, and mutations at the base level compared to a reference sample can be determined.

The term “enrich” as used herein refers to increasing the proportion of a desired substance, for example, to increase the relative frequency of at least one nucleic acid sequence compared to its natural frequency in a high throughput sequencing reaction. Positive selection, negative selection, or both are generally considered necessary to any enrichment scheme. Enrichment methods include, without limitation, hybrid capture enrichment and amplification-based enrichment.

As used herein, the term “gene fusion” or “fusion” refers to a hybrid gene that combines parts of two or more original genes. It can form as a result of chromosomal rearrangements or abnormal transcription. The product of a gene fusion may be a chimeric protein, or the fusion may make the gene non-functional. When expressed, chimeric products can be non-functional, or they can be highly over or underactive. This can cause deleterious effects in cancer such as hyper-proliferative or anti-apoptotic phenotypes.

As used herein, the term “insertions and deletions” or “indels” refers to a variant resulting from the gain or loss of DNA base pairs within an analyzed region.

The term “hybridization” refers to the process of combining complementary, single-stranded nucleic acids into a single molecule. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed.

The term “library” as used herein refers to a collection of DNA molecules which, together, commonly represents the entire genome of an organism (a whole genomic library), or part of the genome of an organism, for example obtained by hybrid capture (a capture library). A library may also consist of a collection of cDNA molecules derived from a mixture of polyadenylated mRNAs isolated from a population of cells of a given strain. Such library representing the transcribed genes of an organism may represent the whole transcriptome or part of the transcriptome following hybrid capture.

The term “ROC” is used herein to refer to a Receiver Operating Characteristic or ROC curve. A ROC curve is a graph showing the performance of a classification model at all classification thresholds. It plots two parameters, which can be true positive fraction (TPF) or true positive rate (TPR) versus false positive fraction (FPF), false positive rate (FPR), or false positives per kilobase (FP per kb). Classifiers that give curves closer to the top-left corner indicate a better performance. The closer the curve comes to the 45-degree diagonal of the ROC space, which represents random guessing, the less accurate the test. The area under the ROC curve is the AUC value. An AUC value of 0.5 is equivalent to a random prediction, and a value of 1.0 is equivalent to a perfect prediction. In general, an AUC of 0.7-0.8 is considered acceptable, 0.8 to 0.9 is considered excellent, and more than 0.9 is considered outstanding.

### Prior Art Problem to be Solved

Genomic analyses are integral to numerous fields, from environmental and microbial surveys, to crop science, and medicine. In a medical context, analyses of genomes are required to conduct paternity analyses, identify rare genetic diseases, and evaluate risks of rare genetic disorders during genetic counselling, pre-implantation tests, and pre-natal tests. As the genomic make-up of individuals affects the susceptibility and tolerance to certain drugs, genomic analyses are moreover relied upon in the context of pharmacogenomics. Similarly, establishing the mutations underlying somatic diseases, such as cancer, helps predict the efficacy of different treatment options. As a consequence, genomic analyses are becoming increasingly important for multiple subfields of precision medicine.

Accessing genomic information has been strongly facilitated by the advent of high-throughput sequencing (HTS), also known as next-generation sequencing (NGS). While low-throughput methods, such as Sanger sequencing, are still used, many medical assays rely on NGS platforms. Their main advantage resides in the generated data volumes, with one NGS run producing millions of sequencing reads. However, DNA samples must undergo a number of transformation steps to produce a sequencing library that is ready for a given NGS platform. DNA mutations can occur during the library preparation and NGS platforms are also subject to sequencing errors. Accurate NGS assays must therefore discriminate real genetic variants, present in the original sample, from artifacts resulting from the library preparation or sequencing.

A number of methods have been developed to discriminate real variants from noise originating during the library preparation and sequencing. A widespread solution consists in calling genetic variants only if the observed variant frequency exceeds a threshold corresponding to that expected in the absence of variant in the sample, also known as background noise. In this context, the ability to detect variants depends on their frequency, as variants representing only a small fraction of the reads covering a given position might be difficult to distinguish from noise. Such variants are expected especially when the sample represents a mixture of cells with and without the variant. In the context of cancer, a biopsy sample will often combine tumorous and non-tumorous cells, in a proportion that is often unknown. In addition, subclonal variants will be restricted to part of the tumor, lowering further their frequency in the sample. Following therapy, the ability to detect low-frequency variants is also crucial to assess measurable residual disease. It is therefore primordial to accurately detect variants at low frequency despite the presence of noises.

Liquid biopsy (analysis of bodily fluids, such as blood plasma) offers a non-invasive method to access tumor DNA. Indeed, DNA, including DNA of tumors, is shed by cells in the bloodstream during apoptosis. The shed DNA, in the form of cell-free DNA (cfDNA), can then be collected far from the tumor site, through a simple blood draw. However, cfDNA is typically present at low concentration, among which the tumor DNA can represent only a small fraction. In addition, blood analyses capture many mutations that are either of germline origin (and therefore present in all cells of the body) or representing somatic mutations originating from the bone marrow and therefore unrelated to the tumor (clonal hematopoiesis of indeterminate potential; CHIP). Gaining insights from liquid biopsy therefore requires methods to confidently detect mutations present at low frequency combined with an ability to differentiate tumor mutations from germline and CHIP variants.

Cell-free DNA is also used in pre-natal genetic tests, as DNA shed by the embryo can be detected in the blood of the mother, providing non-invasive access to the embryo genome. Embryo cfDNA will however represent a small fraction of the DNA isolated from the mother blood, requiring method to identify low-frequency genetic variants.

In the context of pre-implantation genetic tests, genetic analyses are conducted on an extremely restricted number of cells. Such tests therefore require the ability to accurately detect variants from a handful of starting cells.

The amount of information available to variant callers can be increased by sequencing more molecules. However, the amount of starting material might be limited, as in the case of tissue biopsies or pre-implantation genetic tests. In addition, sequencing reads are not statistically independent if they are derived from the same molecule in the original sample. Indeed, the PCR-based amplification that is part of many library preparation protocols creates copies of the original molecules. In the case of limited starting material, producing a large number of sequencing reads will therefore replicate numerous times the same information. Reads originating from distinct molecules can be identified based on the start and end positions of the reads, or pairs of reads, with respect to the reference genome. In addition, molecular identifiers can be integrated during the library preparation to tag individual molecules. The molecular identifiers thereby act by increasing the diversity of sequences at the beginning and end of the reads or pairs of reads. A large number of distinct molecular identifiers can be used, increasing the probability that each molecule is associated to distinct identifiers. Alternatively, the number of distinct identifiers can be limited. Associated with start-end positions, molecular identifiers can in all cases can be used to help identify reads stemming from distinct original molecules. The different approaches to confidently ascertain that some reads originate from distinct original molecules are described as unique molecular identifiers or UMIs (Kivioja et al. 2012, “Counting absolute numbers of molecules using unique molecular identifiers”, Nature Methods 9, 72-74).

Variant calling in the presence of UMIs commonly uses consensus approaches. For quantitative measures, such as copy-number variants or gene expression variants, the most common approach consists in counting the number of distinct UMIs, without considering the number of reads belonging to each UMI group. For the detection of single-nucleotide variants (SNVs) and short insertion/deletion (indels), the consensus approach consists in generating one consensus sequence per UMI, using one of the consensus types known in the art. In some cases, UMIs are used to recognize reads, or pairs of reads, stemming from each of the two strands of the original molecule, and a consensus can be constructed first per strand and then among the two strands (Schmitt et al. 2012, “Detection of ultra-rare mutations by next generation sequencing”, Proc Natl Acad USA 109, 14508-14513). Such approaches however reduce the available information, such as the exact fraction of reads within a given UMI groups having each variant, the phased variants at the read level, the quality of individual reads, and the like. Alternative approaches are therefore needed to fully use the whole extent of information.

Thus, there remains a need for improved methods of generating a DNA library which can be coupled to integrated low-frequency variant identification, possibly independently from an explicit molecular barcoding consensus sequencing error identification/correction, e.g., by tracking both strands of a duplex DNA sample (such as genomic DNA fragments or cfDNA fragments) to detect very low frequency mutations and polymorphisms. For example, there remains a need for efficient and reliable methods of detection of rare or low-frequency mutations and polymorphisms in cancer cells, chimeric cells, and other forms of intra-subject genetic polymorphisms. There also remains a need for improved methods of generating a DNA library which methods may track both strands of the same DNA molecule and facilitate the identification and reporting of multiple low frequency variants without the need for explicit consensus sequencing. There also remains a need for improved methods of producing asymmetric fragmented DNA libraries having different properties of the sequences on each end of a DNA fragment to be sequenced or analyzed.

### Workflow

The starting materials (e.g., DNA) are processed to produce a sequencing library. In the case of genomic DNA (gDNA), the DNA is first fragmented to decrease the size of DNA molecules, using enzymatic fragmentation or physical fragmentation. In the case of cfDNA or degraded genomic DNA, no fragmentation is performed as the DNA is already present as short molecules. In the case of RNA (including cfRNA), the RNA is first reverse transcribed into complementary cDNA using a reverse transcription reaction. The resulting cDNA may not be subjected to fragmentation.

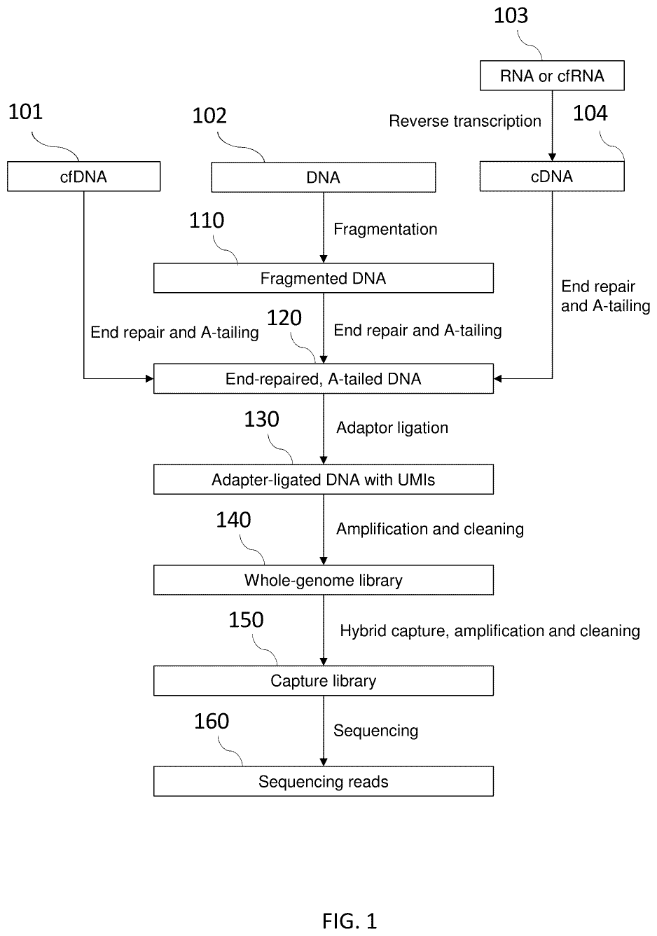

FIG. 1 represents the overall workflow to prepare a sequencing library from different starting materials 101-104. In some embodiments, if the starting material is cfDNA 103, the starting material is directly used 120 as input for end repair and A-tailing. In some embodiments, if the starting material is DNA 102, for example genomic DNA, the starting materials are fragmented to produce fragmented DNA 110. In some embodiments, if the starting material is RNA and/or cfRNA 103, the RNA and/or cfRNA are reverse transcribed to cDNA 104.

In some embodiments, the ends of DNA fragments are repaired 120 to produce blunt-ended double-stranded DNA fragments. A 3′ A-overhang is added 120 to facilitate ligation. Partially double-stranded Y-shaped or U-shaped adapters comprising unique molecular identifiers (UMIs) are then ligated to each end of the DNA fragments to produce adapter-ligated DNA with UMIs 130. In some embodiments, the adapters contain a T 3′ overhang to ligate to the A 3′ overhangs of the DNA fragments. In some embodiments, the adaptors contain primer binding sites in the single-stranded portion. In some embodiments, other types of adapters and other ligation methods are used. In some embodiments, the adaptors contain a variable length sequence terminated by a terminal subsequence at the end of the double-stranded portion. A mixture of different variable length sequences, truncated from a subsequence S, are used, so that different variable length sequences may be ligated among ends and among molecules. In some embodiments, the adapters contain other types of molecular identifiers.

In some embodiments, the adaptor ligated DNA is amplified and cleaned to produce a whole-genome library 140. A PCR amplification is used to generate copies of DNA fragments with adapters ligated on both ends. The PCR primers may incorporate in the adapters the primers used for sequencing, and universal dual indexes, used to recognize samples after multiplexing. In some embodiments, the priming sequences and indexes might be inserted differently. After cleanup, the resultant is a whole-genome sequencing library 140. In some embodiments, the library may be subjected to high-throughput sequencing to produce whole-genome sequencing reads. In some embodiments, the whole genome library 140 is enriched using a target enrichment technique (e.g., hybrid capture), amplified, and cleaned to produce a capture library 150. In some embodiments, the whole-genome sequencing library is subjected to target enrichment. In the embodiments where paired gDNA and cfDNA are analyzed jointly, the prepared gDNA and cfDNA libraries, which incorporate distinct unique dual indexes, are pooled, with varying proportions so that the cfDNA library is more represented than the gDNA library. In some embodiments, the capture library 150 is sequenced to produce sequencing reads 160.

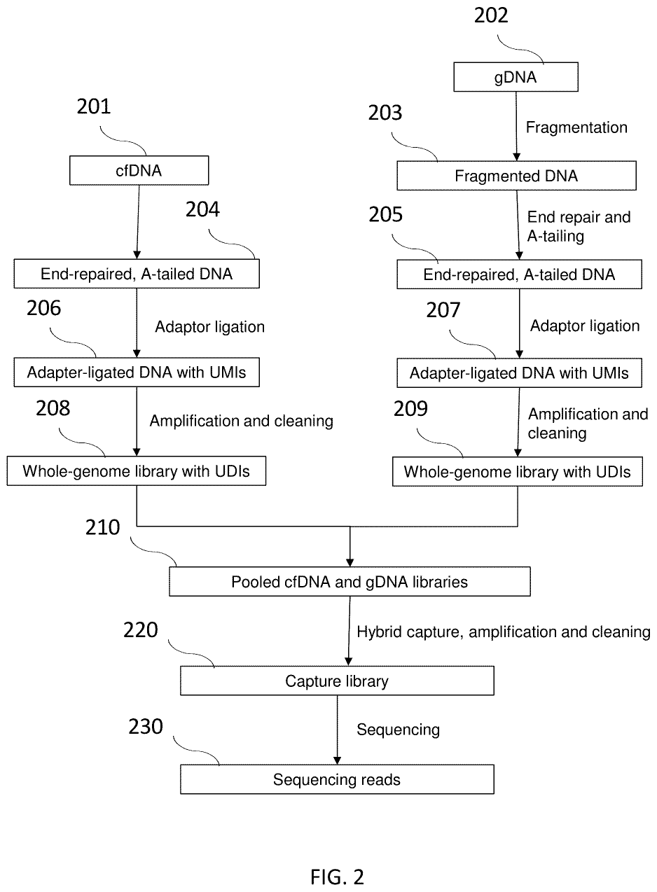

FIG. 2 illustrates a workflow to analyze in parallel paired cfDNA and gDNA. In some embodiments, the starting materials are cfDNA 201 and gDNA 202 analyzed in parallel. In some embodiments the gDNA 202 is fragmented to produce fragmented DNA 203. In some embodiments, the cfDNA 201 and fragmented gDNA 203 are end-repaired and A-tailed 204-205. In some embodiments, the end-repaired and A-tailed DNA 204-205 are ligated to adapters, as described herein to produce adapter-ligated DNA with UMIs 206-207. In some embodiments, the adapter-ligated DNA is amplified and cleaned to produce a whole-genome library 208-209. In some embodiments, unique dual indexes (UDIs) are integrated in the whole genome sequencing library 208-209 differently tag the molecules originating from different samples. In some embodiments, different UDIs are used for the cfDNA whole-genome sequencing library 208 and the gDNA whole-genome sequencing library 209. In some embodiments, the gDNA and cfDNA whole-genome libraries 208-209 are pooled 210. It will be apparent to those skilled in the art that the paired cfDNA and gDNA can be pooled after the hybrid capture step or can be sequenced independently without changing the invention.

In some embodiments, a plurality of genomic regions are selected from the DNA preparation by hybridizing DNA fragments to biotinylated capture probes. In some embodiments, the capture probes are designed to hybridize to genomic regions containing sites relevant for inherited disorders. In some embodiments, the capture probes are designed to hybridize to genomic regions of relevance for a large array of cancer types. In some embodiments, the capture probes are designed to hybridize to genomic regions of relevance for specific cancer types.

In some embodiments, the capture probes are designed to hybridize to genomic regions corresponding to the genes AKT1, ALK, APC, AR, ARAF, ARID1A, ARID2, ASXL1, ATM, B2M, BAP1, BCL2, BCOR, BRAF, BRCA1, BRCA2, CARD11, CBFB, CBL, CCND1, CD79B, CDH1, CDK12, CDK4, CDKN2A, CHEK2, CIC, CREBBP, CTCF, CTNNB1, DICER1, DIS3, DNMT3A, EGFR, EIF1AX, EP300, ERBB2, ERBB3, ERCC2, ESR1, EZH2, FBXW7, FGFR1, FGFR2, FGFR3, FGFR4, FLT3, FOXA1, FOXL2, FOXO1, FOXP1, FUBP1, GATA3, GNA11, GNAQ, GNAS, H3F3A, HISTIH3B, HRAS, IDH1, IDH2, IKZF1, INPPL1, JAK1, JAK2, KDM6A, KEAP1, KIT, KNSTRN, KRAS, MAP2K1, MAP2K2, MAPK1, MAX, MED12, MET, MLH1, MSH2, MSH3, MSH6, MTOR, MYC, MYCN, MYD88, MYOD1, NF1, NFE2L2, NOTCH1, NPM1, NRAS, NTRK1, NTRK2, NTRK3, NUP93, PAK5, PALB2, PDGFRA, PHF6, PIK3CA, PIK3CB, PIK3R1, PIK3R2, PIMI, PMS2, POLE, POT1, PPM1D, PPP2R1A, PPP6C, PRKC1, PTCH1, PTEN, PTPN11, RAC1, RAD54L, RAF1, RB1, RET, RHOA, RIT1, ROS1, RRAS2, RXRA, SETD2, SF3B1, SMAD3, SMAD4, SMARCA4, SMARCB1, SOS1, SPOP, SRSF2, STAT3, STK11, STK19, TCF7L2, TET2, TGFBR1, TGFBR2, TP53, TP63, TSC1, TSC2, U2AF1, VHL, XPO1. In some embodiments, other regions are added to this list to capture clinically-relevant mutations.

In some embodiments, the capture probes include probes designed to hybridize DNA segments with subregions originating from two distinct genomic locations, for example following gene fusions and other genomic rearrangements. In some embodiments, the capture probes include probes designed to hybridize DNA segments originating from genomic regions with mono- or oligo-nucleotide repeats, such as microsatellite. These regions may have been selected for their usefulness for the inference of microsatellite instability, for instance based on previous experiments. In some embodiments, the DNA fragments ligated to biotinylated captured probes are isolated using streptavidin-coated magnetic beads. The DNA fragments are then released, washed and PCR amplified. After clean-up, the result is a capture library 220, which can be subjected to high-throughput sequencing to produce sequencing reads 230. It will be apparent to those skilled in the art that variation in the hybrid capture protocol can be inserted without changing the invention. In some embodiments, an aliquot of the whole-genome sequencing library may be mixed with the capture library to provide a low-level coverage of the whole genome in addition to a high-level coverage of targeted regions. In some embodiments, the libraries obtained from different samples, with different indexes, may be pooled. In some embodiments, sequencing-by-synthesis is used to generate paired-end reads. It will be apparent to those skilled in the art that the invention can be modified to work with single-end reads.

If multiple samples were sequenced as part of the same sequencing run, the sequencing reads 230 may be demultiplexed per sample based on the sample indexes. In embodiments where paired gDNA and cfDNA are analyzed jointly, reads corresponding to each sample type may similarly be identified based on their indexes and separated.

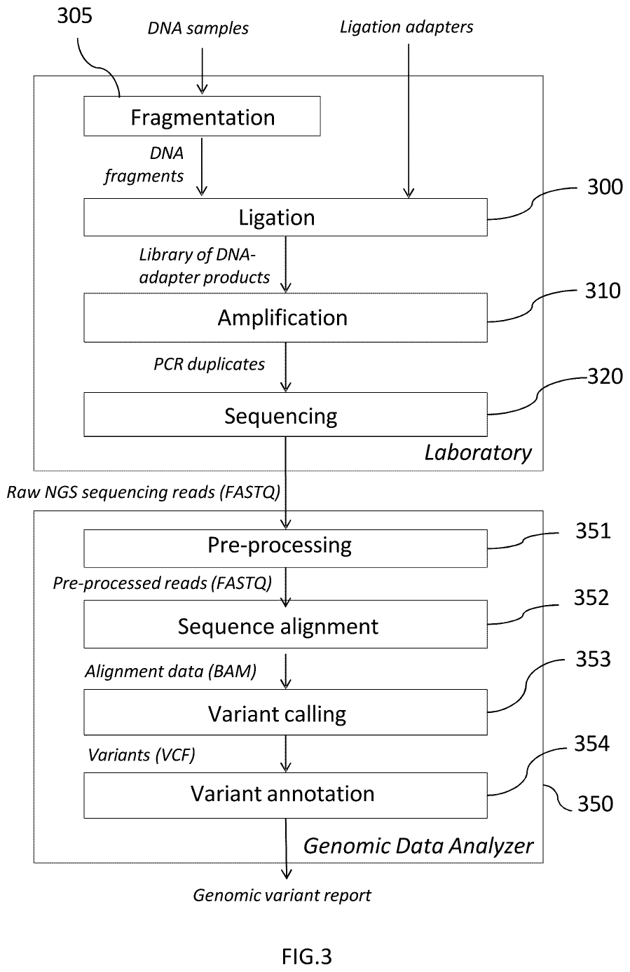

An exemplary low frequency DNA variant identification workflow will now be described with further detail with reference to FIG. 3. As will be apparent to those skilled in the art of DNA analysis, such a workflow comprises preliminary experimental steps to be conducted in a laboratory (also known as the “wet lab”) to produce DNA analysis data, such as raw sequencing reads in a next-generation sequencing workflow, as well as subsequent data processing steps to be conducted on the DNA analysis data to further identify information of interest to the end users, such as the detailed identification of DNA variants and related annotations, with a bioinformatics system (also known as the “dry lab”). Depending on the actual application, laboratory setup and bioinformatics platforms, various embodiments of a DNA analysis workflow are possible. FIG. 3 describes an example of a workflow comprising a wet lab process wherein DNA samples are first fragmented with a fragmentation protocol 305 (optional) to produce DNA fragments. The DNA ends of these DNA fragments are then repaired and modified such as to be compatible with the adaptors that will be used. Adaptors as will be further described in more detail throughout this disclosure may then be joined by ligation 300 to the DNA fragments in a reaction mixture, so as to produce a library of DNA-adaptor products, in accordance with some of the proposed methods. The DNA library further undergoes amplification 310 and sequencing 320. In a next generation sequencing workflow, the resulting DNA analysis data may be produced as a data file of raw sequencing reads in the FASTQ format. The workflow may then further comprise a dry lab Genomic Data Analyzer system 350 which takes as input the raw sequencing reads for a pool of DNA samples prepared with the ligation adaptors according to the proposed methods, and applies a series of data processing steps to identify genomic variants, for instance as a genomic variant report for the end user. An exemplary Genomic Data Analyzer system 350 is the SOPHIA GENETICS Data Driven Medicine platform (SOPHIA DDM) as already used by hospitals worldwide in 2019, but other systems may be used as well. Different detailed possible embodiments of data processing steps as may be applied by the Genomic Data Analyzer system 350 are described for instance in the international PCT patent application WO2017/220508, but other embodiments are also possible.

In an embodiment, the Genomic Data Analyzer system 350 may first apply one or more pre-processing steps 351 to produce pre-processed reads from the raw sequencing reads inputs. The pre-processing steps may for instance comprise adaptor trimming, as well as read sorting, to analyze and group reads in families of reads issued from similar DNA fragments in accordance with the proposed adaptor ligation methods and numerical coding methods as will be further described herein. In a possible embodiment, the raw reads as well as the pre-processed reads may be stored in the FASTQ file format, but other embodiments are also possible.

The Genomic Data Analyzer system 350 may further apply sequence alignment 352 to the pre-processed reads to produce read alignment data. In one embodiment, the read alignment data may be produced for instance in the BAM or SAM file format, but other embodiments are also possible.

The Genomic Data Analyzer system 350 may further apply variant calling 353 to the read alignment data to produce variant calling data. In one embodiment, the variant calling data may be produced for instance in the VCF file format, but other embodiments are also possible.

The Genomic Data Analyzer system 350 may further apply variant annotation 354 to the read alignment data to produce a genomic variant report for each DNA sample. In one embodiment, the genomic variant report may be visualized by the end user on a graphical user interface. In another possible embodiment, the genomic variant report may be produced as a text file for further data processing. Other embodiments are also possible.

### Fragmentation

In some embodiments, methods as described herein will involve the use of genomic and mitochondrial DNA to be sequenced and determination of such information as the location and coding of genes, promoters, exons, introns, and potentially epigenetic information, such as CpG islands, and methylation, potentially in conjunction with bisulfide conversion. Genomic DNA may be chromosomal DNA or circular DNA. Alternately, mRNA may be reverse transcribed into complementary DNA or cDNA, and said cDNA may be fragmented, or it may be of a small enough length that it may be sequenced without fragmentation. The complementary cDNA, fragmented or non-fragmented, may be single-stranded, and could then be made double-stranded by annealing random primers and/or other primers and elongating the primers to be complementary to the cDNA, thus forming a double-stranded cDNA. In some embodiments, the double-stranded cDNA and mitochondrial and/or genomic DNA must be fragmented 50 prior to sequencing 320. Fragmentation 305 may be achieved by several means including but not limited to sonication, ultrasonication, mechanical shearing, partial digestion via for example restriction enzyme digestion, etc. Fragmentation may result in a fragmented DNA being 50 to 10000 base-pairs in length, preferably 200 base-pairs to 800 base-pairs in length, more preferably 300 to 500 base-pairs in length, and more preferably still 400 base-pairs in length. The DNA fragments, whether from cDNA, genomic DNA, cell-free DNA, or mitochondrial DNA, may be sized-fractionated, for example by agarose gel electrophoresis; gel chromatography; equilibrium density-gradient centrifugation, including sucrose gradient centrifugation, percol gradient centrifugation, cesium-chloride centrifugation; and other means.

### Adaptor Ligation/Insertion

After fragmentation and end-repair 305, in the case of genomic DNA or chromosomal DNA or reverse transcription followed by formation of double-stranded DNA, adaptors may be ligated or linked 300 to each of the ends of the fragmented double-stranded DNAs.

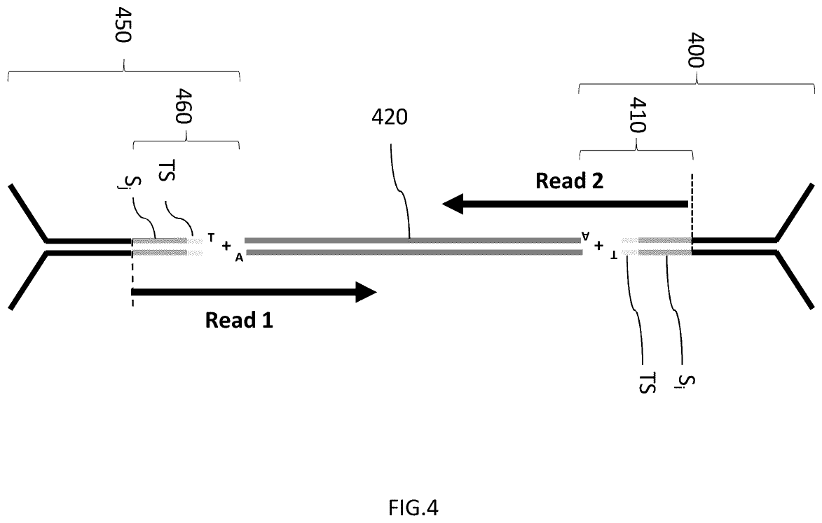

FIG. 4 shows an embodiment of the ligation 300 of two adaptors 400, 450 to each end of a DNA fragment 420. Each adaptor 400, 450 as shown in the exemplary embodiment as illustrated by FIG. 4 may comprise a partially double-stranded molecule of DNA with a single nucleotide (T) 3′ overhang at the end to be annealed to the double-stranded fragmented DNA. Each adaptor 400, 450 comprises a double stranded segment 410, 460 at one end which constitutes a spacer sequence (SS) separating the adaptor 400, 450 from the DNA fragment 420 nucleotide sequences in subsequent high-throughput sequencing reads (Read 1, Read 2). In a possible embodiment as illustrated by FIG. 4, the latter spacer sequence end may contain a single-nucleotide T 3′ overhang, but other embodiments are also possible as will be apparent to those skilled in the art, for instance it may be blunt ended or it may be substituted by another 3′ or 5′ overhang, so as to facilitate the ligation 300 of the adaptor 400, 450 to the target double stranded DNA molecules 420 (e.g., genomic DNA or “gDNA”).

An adaptor comprises a double-stranded sequence at the end being annealed to the double-stranded DNA. In this regard, one of the two strands of the double-stranded sequences of the adaptor will be ligated to the 3′ end of the fragmented double-stranded DNA, and the other of the two strands of the double-stranded sequences of the adaptor will be ligated to the 5′ end of the fragmented double-stranded DNA.

The ends of the double-stranded sequences of the adaptors being ligated to the fragmented double-stranded DNA are not limited and may comprise blunt ends, 3′ overhangs, and 5′ overhangs. In this regard, the 5′ends of the adaptors being ligated could either terminate with a 5′-phosphate or a 5′-OH. If a 5′-OH is at the adaptor end to be ligated to the target nucleic acid, it may be necessary to use a polynucleotide kinase to complete the backbone and join the 5′-OH of the adaptor to the 3′-OH of the fragmented DNA. In some embodiments a one nucleotide overhang able to be ligated by T-4 ligase from the T-4 bacteriophage is preferable. Thus, in some embodiments, an adenine may be added to each of the 3′ blunt ends of the fragmented DNA prior to adaptor ligation, and the adaptor may have a thymidine overhang on the 3′ end to base-pair with the adenine added to the 3′ end of the fragmented DNA. In some embodiments, an adenine may be added to each of the 3′ blunt ends of the fragmented DNA prior to adaptor ligation, and the adaptor may have a phosphorothioate bond before the terminal thymidine on the 3′ end to base-pair with the adenine added to the 3′ end of the fragmented DNA. The phosphorothioate bond before the terminal thymidine will prevent an exonuclease from trimming the thymidine, thus creating a blunt end when the end of the adaptor being ligated is double-stranded.

Adaptors with Variable Length Spacer Sequence

In an embodiment, as illustrated on FIG. 4, each adaptor 400, 450 comprises a spacer sequence 410, 460 terminating its double-stranded end to be linked to the DNA fragment 420. In one embodiment, part or all of the spacer sequence 410, 460 may be truncated from a predefined, constant nucleotide sequence S of length L_{S} nucleotides to form a diversity of variable length truncated spacer subsequences Sᵢ, Sⱼ.

In one embodiment, in order to facilitate downstream read trimming pre-processing 351 by the genomic data analyzer 350 out from the raw sequencing reads, each truncated spacer subsequence Sᵢ, Sⱼ of respective lengths L_{Si}, L_{Sj} of at most L_{S} nucleotides may be followed by a constant termination subsequence TS of a length L_{TS} of at least 3 nucleotides, for instance by concatenating each truncated variable length subsequence Sᵢ, Sⱼ with the TS termination subsequence, to form the variable length spacer sequences 410, 460, as illustrated on FIG. 4.

Preferably, the predefined, constant nucleotide sequence S and the constant termination subsequence TS are chosen such that the constant termination subsequence TS differs from the reminder of the sequence S by an edit distance of at least two. As illustrated by FIG. 4, each adaptor spacer sequence SS 410, 460 may thus be terminated by the same, constant termination subsequence TS of at least 3 nucleotides, the termination subsequence TS differing from the reminder of the sequence S (and thus from any of its derived truncated spacer subsequences Sᵢ, Sⱼ) by an edit distance of at least two.

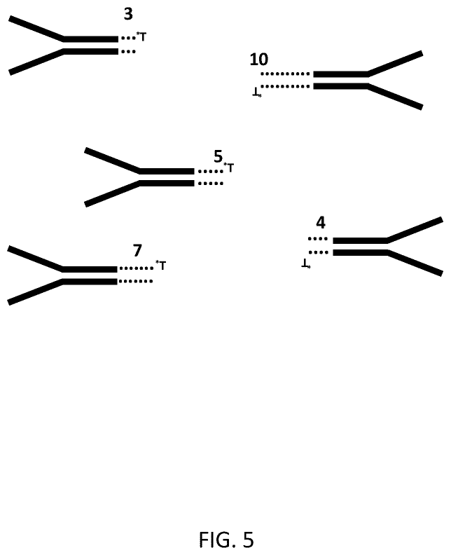

As illustrated by FIG. 5, a plurality of adaptors may be used, differing from each other specifically by the length of their truncated spacer subsequence Sᵢ, Sⱼ. The resulting total length for the spacer sequence 410, 460 once concatenated with a constant termination subsequence TS may thus be for example 3 nucleotides (as is the minimal size for a termination subsequence which may be used as a “triplet stop code” to facilitate downstream read trimming pre-processing 151), 10 (7+3) nucleotides, 5 (2+3) nucleotides, 7 (4+3) nucleotides, 4 (1+3) nucleotides . . . . More generally, the spacer sequence variable length may be at least L_{TS}=3 nucleotides, and at most Lₘₐₓ=L_{S}+L_{TS} nucleotides. Similarly, for a quadruplet termination subsequence TS, the spacer sequence variable length may be at least L_{TS}=4 nucleotides, and at most Lₘₐₓ=L_{S}+L_{TS} nucleotides, etc.

In general, the maximal length L_{S} of the constant polynucleotide sequence S may be chosen such that the derived spacer sequence 410, 460 does not take too long a segment relative to the total sequencing read length (which may be as low as 350 base pairs in some high throughput sequencing workflows) while enabling enough different variable truncated lengths to provide the required combinatorial diversity to discriminate PCR duplicates from similar DNA fragments from the bioinformatics workflow point of view, that is fragments which share the same reference mapping positions once ligated with a couple of adaptors out of the plurality of adaptors with different truncated lengths. In a possible embodiment, L_{S} may be chosen as 5, 6, 7, 8, 9, 10, 11, 12, 13, 14, 15, 16, 17, 18, 19 or 20 nucleotides, but other embodiments are also possible. When preparing a pool of samples for high throughput sequencing, in a possible embodiment the same constant polynucleotide sequence S may be used to prepare the ligation adapters for all samples; in an alternate embodiment different constant polynucleotide sequences may be defined and used to prepare the ligation adapters for different samples.

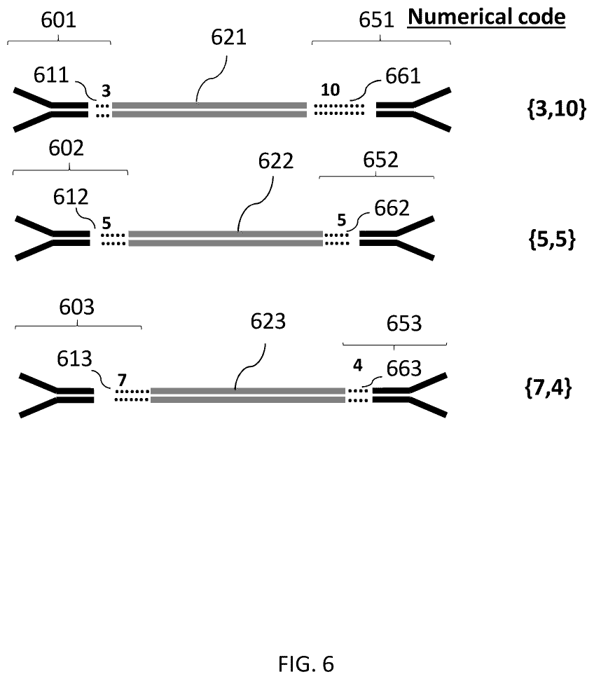

FIG. 6 illustrates the resulting ligation and corresponding numerical code for three exemplary DNA fragments 621, 622, 623. The first DNA fragment 621 is ligated to a first adaptor 601 comprising a spacer sequence 611 (SS₁) having a total length L₁ of 3 nucleotides on one end and to a second adaptor 651 comprising a spacer sequence 661 (SS₂) having a total length L₂ of 10 nucleotides on its other end. The second DNA fragment 622 is ligated to a third adaptor 602 comprising a spacer sequence 612 (SS₃) having a total length L₃ of 5 nucleotides on one end and to a fourth adaptor 652 comprising the same spacer sequence 662 (SS₄-note that in this specific example SS₄=SS₃) having a total length L₄=L₃₌₅ nucleotides on its other end. The third DNA fragment 623 is ligated to a fifth adaptor 603 comprising a spacer sequence 613 (SS₅) having a total length L_{S} of 7 nucleotides on one end and to a sixth adaptor 653 comprising a spacer sequence 663 (SS₆) having a total length L₆=4 nucleotides on its other end. The first DNA-adaptor product issued from DNA fragment 621 may thus be associated with a numerical code {3,10} (or {10,3} depending on the read direction) corresponding to the respective lengths of the spacer sequences from its adaptors on both ends. The second DNA-adaptor product issued from DNA fragment 622 may thus be associated with a numerical code {5,5} (in any read direction) corresponding to the respective lengths of the spacer sequences from its adaptors on both ends. The third DNA-adaptor product issued from DNA fragment 623 may thus be associated with a numerical code {7,4} (or {4,7} depending on the read direction) corresponding to the respective lengths of the spacer sequences from its adaptors on both ends. It is thus possible to discriminate between the first, second and third DNA-adaptor products with identical mapping positions in a DNA library and to trace back the derivative DNA products from each parent DNA-adaptor product by identifying the spacer sequences on both ends of the derivative DNA products and measuring their respective lengths to identify the numerical code inherited from the parent DNA-adaptor product.

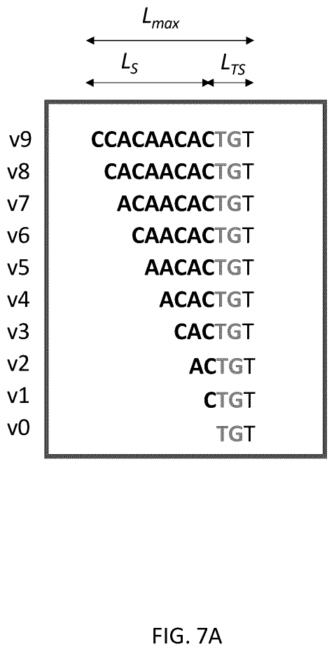

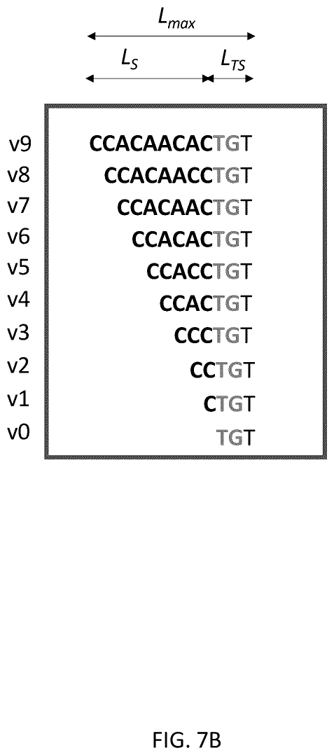

FIG. 7A illustrates a first example of ten possible spacer sequences identified in FIG. 7A as v9, v8, v7, v6, v5, v4, v3, v2, v1, v0. Each spacer sequence may be formed by the truncation left to right from the start of a constant sequence S=CCACAACAC of length L_{S}=9, further concatenated with a termination subsequence (TS) triplet T,G,T (itself ending with a T overhang to facilitate the ligation). FIG. 7B illustrates an alternate, second example of ten possible spacer sequences which may be truncated right to left from the end of a constant sequence S=CCACAACAC of length L_{S}=9, further concatenated with a termination subsequence (TS) triplet T,G,T (itself ending with a T overhang to facilitate the ligation). In both examples of FIG. 7A and FIG. 7B, the constant sequence S=CCACAACAC is of length L_{S}=9 and each possible derived truncated subsequence has a subsequence length of 9, 8, 7, 6, 5, 4, 3, 2, 1 and 0 nucleotide respectively. When followed by a triplet of T,G,T nucleotides, which will correspond to a triplet code TGT in the resulting sequencing reads, the resulting spacer sequence total lengths are then respectively 12, 11, 10, 9, 8, 7, 6, 5, 4, and 3 nucleotides.

In a possible embodiment, the full spacer sequence length of the truncated plus termination subsequences (absolute length, for instance numerical in the range values 3 to 12) may be used to form the numerical code. In an alternate embodiment the sole length of the truncated subsequence part of the spacer sequence, thus excluding the constant length of the termination subsequences, may be used to form the numerical code (relative length, for instance numerical values in the range 0 to 7).

### Amplification and Sequencing

Once the DNA products have been produced with adaptor ligation, they may be amplified by a polynucleotide amplification reaction to produce multiple polynucleotide sequences replicated from one or more parent sequence. As will be apparent to those skilled in the art of next generation sequencing, amplification may be produced by various methods, for instance a polymerase chain reaction (PCR), a linear polymerase chain reaction, a nucleic acid sequence-based amplification, rolling circle amplification, and other methods. In some embodiments, after library amplification, DNA-adaptor products can then be sequenced using any technology known in the art including, but not limited to, the Illumina sequencing technology, the Ion Torrent sequencing technology, the 454 Life Sciences sequencing technology, the ABI SOliD sequencing technology, the Pacific Biosciences sequencing technology or the Oxford nanopore sequencing technology. For example, in the case of the Illumina sequencing platform, the sequencer primering sequences present on both ends of library products have the functional property of annealing or binding to the flow cell oligomers or flow cell sequences. As will be apparent to those skilled in the art of next generation sequencing, a bridge-amplification process may then be carried out wherein the fragmented DNA comprising the adaptor sequences (including the spacer sequences), the first primering sequences, and the second primering sequences will be annealed to either a first and/or second immobilizer sequences. The 3′-OH of the first and/or second immobilizer sequences will then be elongated, using the fragmented DNA comprising the adaptor sequences, the first primering sequence, and the second primering sequence, as a template—the genetic information within the fragmented DNA comprising the adaptor sequences (including the proposed spacer sequences), the first primering sequence, and the second primering sequence will thus be transferred to the first or second immobilizer sequences and thus bound to the solid state support. The fragmented DNA comprising the adaptor sequences (including the proposed spacer sequences), the first primering sequences, and the second primering sequences will then be denatured or deannealed and removed. The bound fragmented DNA will then be annealed to the immobilizer sequence at the free end of the bound fragmented DNA and undergo several cycles of bridge amplification.

At this point in time, the cluster generation process has been completed and the flow cell is configured in such a way as to permit sequencing by synthesis by the reannealing of the free immobilizing sequence to the cleaved and therefore free immobilizer sequence. After priming, each nucleotide may be incorporated into the newly synthesized strand of DNA based on the template strand annealed to the solid state support during cluster generation. Each nucleotide being incorporated into the newly synthesized strand is associated with a different fluorophore, and each fluorophore may emit a different wave-length of light when the newly incorporated nucleotide may be integrated into the new strand of DNA and/or base-pairs with its complementary counterpart (A to T, G to C) during elongation.

In exonuclease based nanopore sequencing the nucleic acid may be digested and the produced free nucleotides will be identified by their effect on the electric potential across a lipid a membrane. A single stranded nucleic acid strand might also be forced to pass through a nanopore driven by differences in the electric potential or assisted by enzymes such helicases or a polymerases. The movement of the nucleic acid strand through the nanopore may produce a change in electric potential allowing the identification of the nucleic acid sequence.

The index sequence may then be used to identify the samples of the sequences. After read pre-processing 351 and read alignment 352, PCR duplicates can be identified using the DNA fragment endogenous information and/or mapping positions, the DNA fragment exogenous mapping positions or a combination thereof to distinguish true mutants from misinsertions caused after the DNA fragmentation.

In some embodiments, during the polymerization or elongation of a new strand of DNA from a template strand, DNA polymerase will sometimes incorrectly position a base which does not base-pair with the nucleotide opposite it on the other strand of DNA—this is referred to as a mismatch or misinsertion. In this regard, the newly synthesized strand of DNA may be considered to be complementary to the template strand, even though one or several mismatches may occur. In embodiments, it is contemplated that this mismatching error by DNA polymerase may occur in a daughter strand of DNA, and that tracking all the copies belonging to the same PCR duplicate group as this daughter strand may permit discrimination of these mismatches from genetic polymorphisms (e.g. mutations) found in the genomic DNA as extracted from the cell.

### Read Pre-Processing

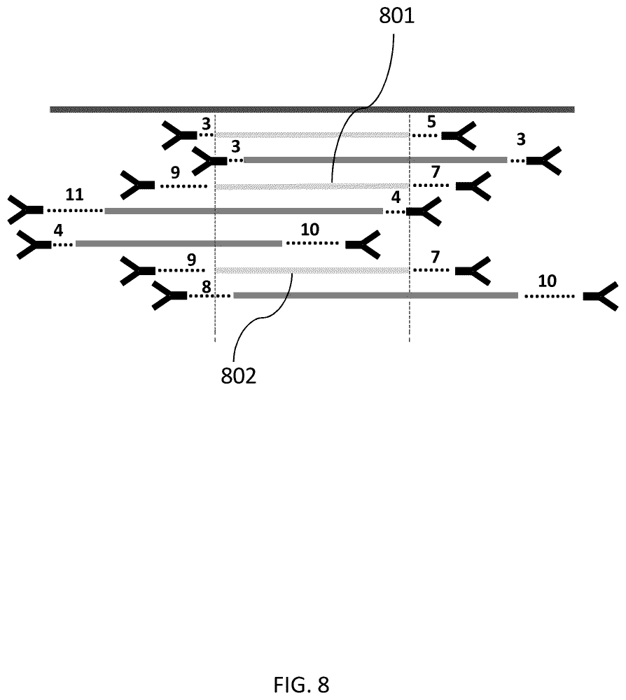

After amplification 310, each DNA-adaptor product is replicated in a plurality of PCR duplicates. As illustrated on FIG. 8, two PCR duplicates 801, 802 issued from the same DNA-adaptor product, that is from the same DNA fragment, will thus carry the same spacer sequences on their ends, which will be found in the resulting raw sequencing reads after sequencing 320. It is therefore possible to group them together in the downstream genomic analysis workflow by measuring the respective lengths of their spacer sequences (numerical code={9,7} in the example of FIG. 8).

As will be appreciated by those skilled in the art of low frequency DNA analysis, PCR duplicates issued from other DNA-adaptor products, that is from different DNA fragments, are unlikely to carry the same spacer sequences lengths provided that 1) the number of different possible adaptor combinations is large enough relative to the number of possibly colliding DNA fragments to discriminate out of the reads with the same start and end positions after alignment 352 and 2) the PCR amplification and sequencing errors, including possible insertion or deletion of nucleotides in the spacer sequence, can be detected thanks to the use of a constant sequence S as the basis for the truncated spacer subsequences to be retrieved in the reads.

As illustrated on FIG. 4, in the case of pair-end sequencing technology, after sequencing two different read directions Read 1 and Read 2 may each generate a different spacer sequence yet with a common termination sequence TS in the FASTQ file, but this spacer sequence may have a different length for each DNA-adaptor product, thus enabling to statistically distinguish it from another one. In the alignment 352 step, the start and the end positions of the DNA fragment sequence 420 to analyze will be thus shifted apart among most of the reads issued from different DNA-adaptor products, thus creating further endogenous diversity.

For instance, with reference to FIG. 6, for the first DNA fragment 621 a first spacer sequence 611 will constitute the first 3 nucleotides in the raw sequencing reads for PCR duplicates read from 3′ to 5′ direction, while the second spacer sequence 661 will constitute the first 10 nucleotides in the raw sequencing reads for the same PCR duplicates read in the reverse 5′ to 3′ direction. For the second DNA fragment 622 the third spacer sequence 612 will constitute the first 5 nucleotides in the raw sequencing reads for PCR duplicates read from 3′ to 5′ direction, and the fourth spacer sequence 662 will constitute the first 5 nucleotides in the raw sequencing reads for the same PCR duplicates read in the reverse 5′ to 3′ direction. For the third DNA fragment 623 the fifth spacer sequence 613 will constitute the first 7 nucleotides in the raw sequencing reads for PCR duplicates read from 3′ to 5′ direction, and the sixth spacer sequence 663 will constitute the first 4 nucleotides in the raw sequencing reads for the same PCR duplicates read in the reverse 5′ to 3′ direction. It is thus possible to uniquely associate a numerical code to each DNA fragment: the combination {L₁, L₂}={3, 10} of the spacer sequence length values for respectively the first end and the second end of the first DNA fragment 621; the combination {L₃, L₄}={5, 5} of the spacer sequence length values for respectively the first end and the second end of the second DNA fragment 622; the combination {L_{S}, L₆}={7, 4} of the spacer sequence length values for respectively the first end and the second end of the third DNA fragment 623, etc. As illustrated by FIG. 6, it is thus possible to group the PCR duplicate raw sequencing reads based on the variable length of the spacer sequences as can be retrieved from their start sequence of nucleotides in the raw sequencing reads sequenced from the PCR duplicates issued from the DNA-adaptor products generated with the proposed method.

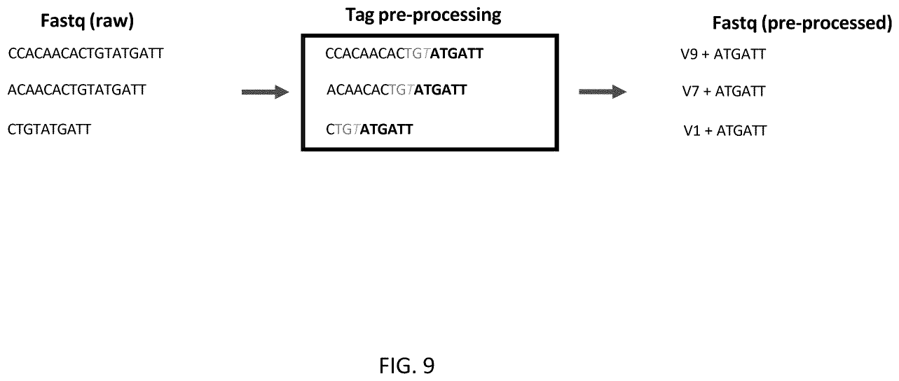

FIG. 9 provides examples of the start sequences for three different reads as may be issued from the sequencing of the DNA-adaptor products constructed according to the exemplary sequences of FIG. 7A). Each spacer sequence ends with the termination sequence triplet TGT as in the example of FIG. 7B), so it is possible for the Genomic Data Analyzer 350 to search for this triplet as part of the read pre-processing step 351.

In a first possible embodiment (not illustrated), the read pre-processing 351 consists in first trimming the read start sequence by Lₘₐₓ nucleotides, Lₘₐₓ being the sum of the length L_{S} of the constant sequence S out from which the subsequences are truncated and of the length L_{TS} of the concatenated constant termination subsequence TS. After trimming the reads in the FASTQ file the remainder of the sequence for each read may be stored in a pre-processed FASTQ file.

As will be apparent to those skilled in the art of sequencing, due to the use of variable length adaptors, the resulting pre-processed reads will be shifted relative to each other with different start and end positions after subsequent alignment 352, which will de facto statistically separate the alignment results issued from different DNA-adaptor products. The latter “endogenous” length discrimination may however not be statistically sufficient to discriminate between the DNA fragments to be analyzed, depending on the actual application needs. Moreover, it has the drawback of losing a few nucleotides at the start of the fragment due to trimming to the length of the longest possible adaptor even for the reads carrying shorter truncated spacer subsequences. Therefore, in alternate embodiment, the termination subsequence TS may be searched at the start of each read sequence. Once it is found, the length of the spacer sequence string may be measured, for instance as the distance between the start of the read and the start of the termination subsequence TS (relative spacer sequence SS length).

Alternately, it may be measured as the distance between the start of the read and the end of the termination subsequence TS (absolute spacer sequence SS length). Each read may thus be assigned a different spacer sequence length measurement as part of the read pre-processing 351 step. In the example of FIG. 9, the first read carries at the beginning the spacer sequence SS₁=CCACAACACTGT of absolute length L₁=12; the second read carries the spacer sequence SS₂=ACAACACTGT of absolute length L₂=10, and the third read carries the spacer sequence SS₃=CTGT of absolute length L₃=4. The measured length value may thus be recorded in the pre-processed FASTQ file so as to provide an extra numerical information enabling to trace back the DNA-adaptor product origin of the read in the downstream alignment process 352. Thus, depending on the actual needs of the application, the read sequence remainder to be input to the alignment may be either generically trimmed to the length Lₘₐₓ of the longest possible spacer sequence, so as to provide a further “endogenous” length discrimination to the alignment process (yet at the expense of losing a few nucleotides at the beginning of the fragment sequence itself), or alternately it may be individually trimmed to the actual spacer sequence SS length Lₙ as measured for each sequencing read Rn by the pre-processing 351 (until the end of the termination sequence TS).

In some embodiments, the variable length spacer may be identified from within each sequencing read based on the presence of the terminal sequence. For each pair of paired-end reads, the length of the variable length spacer attached on each side is then recorded, starting with that of the read in the same orientation as the reference genome. The variable length spacers are then truncated from each read. In embodiments involving other types of molecular identifiers, their identification may rely on sequences known a prior or on their expected length. In all embodiments, remaining adapter sequencing may be identified and trimmed. Low quality reads may similarly be truncated or discarded.

### Read Mapping and Alignment

The resulting pre-processed reads may then be aligned 352 to a reference genome. It is then possible to discriminate in the data records (typically stored as BAM or SAM file formats) the set of reads issued from PCR duplicates of different original DNA fragments based on one or more of the following features available in the data records:
1) The numerical code obtained by combining the adaptor spacer sequence lengths measured in the reads;
2) The mapping position (i.e., start-end) of the DNA fragment, relative to the reference genome.

In the case of pair-end sequencing, the pair-end read orientation information (i.e., FIR2 or F2R1), which allows to discriminate pair-end reads issued from the original plus or minus strand may be used. For each couple of pair-end reads (i.e., R1 and R2) it is possible to recover their possibly different adaptor lengths and use these numbers to form a numerical code (composed of a pair of integer values) to be stored as a tag in a read alignment file, such as a BAM format file. In a first step, pair-end reads aligned to the same start and end position (relative to the reference genome sequence reading direction) and having the same pair of measured adaptor lengths (L1, L2) or (L2, L1) may be grouped as sequencing reads possibly issued from the two strands of the same original double-stranded DNA fragment. Then each group may be further subdivided in two sub-groups according to their strand of origin, where the actual pair of measured adaptor lengths (L1, L2) is given by {L_{n (forward)}, L_{m (reverse)}} in case of pair-end reads with F1R2 orientation and by {L_{n (reverse)}, L_{m (forward)}} in case of pair-end reads with F2R1 orientation.

The resulting information may be recorded in a raw fragment-tagged read alignment file, such as a BAM or SAM format file. Using this file, it is possible to cluster groups of pair-end reads issued from the same fragment ligation from the alignment, so that downstream genomic analysis steps such as variant calling 353 can be performed by exploiting the information provided by PCR duplicates issued from the two strands of the original DNA fragment.

In some embodiments, the reference genome is a complete human genome. In some embodiments, the global alignment is refined locally. In some embodiments, multiple alignment is performed across reads mapping to the same genomic region to reconcile putative insertions and deletions. In embodiments involving hybrid capture target enrichment, reads overlapping targeted genomic regions are retained.

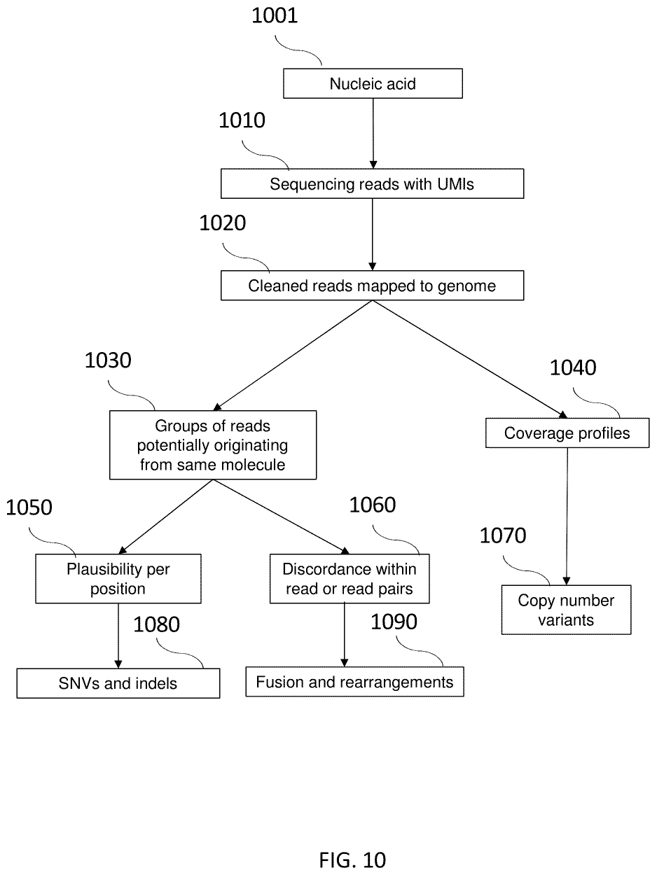

As illustrated in FIG. 10, the analyses leading to the identification of different types of genetic variants can differ. For example, in some embodiments, nucleic acids 1001 are sequenced according to the methods described herein to produce sequencing reads with unique molecular identifiers 1010. In some embodiments, the reads are cleaned and mapped to a reference genome 1020. As described above, the pre-processing to obtain cleaned reads may involve generating numerical codes representing the length of the space length adapters. In some embodiments, groups of reads potentially originating from the same molecule 1030 are determined from the cleaned and mapped sequencing reads 1020. As described above, the identification of read groups may rely on a combination of numerical codes representing the space length adapters and the start-end positions of the mapping. In some embodiments, plausibility per position 1050 are calculated for the groups of reads potentially originating from the same molecule 1030 and are incorporated to identify SNVs and insertions/deletions (indels) 1080. In some embodiments, discordance within reads or read pairs 1060 are determined for the groups of reads potentially originating from the same read groups 1030 and fusions and chromosomal rearrangements 1090 are determined. It will be apparent to those skilled in the art that fusions and chromosomal rearrangements 1090 may be inferred directly from the cleaned reads mapped to the genome 1020. In some embodiments, coverage profiles 1040 are determined from the cleaned and mapped sequencing reads 1020 and used to infer copy number variants 1070.

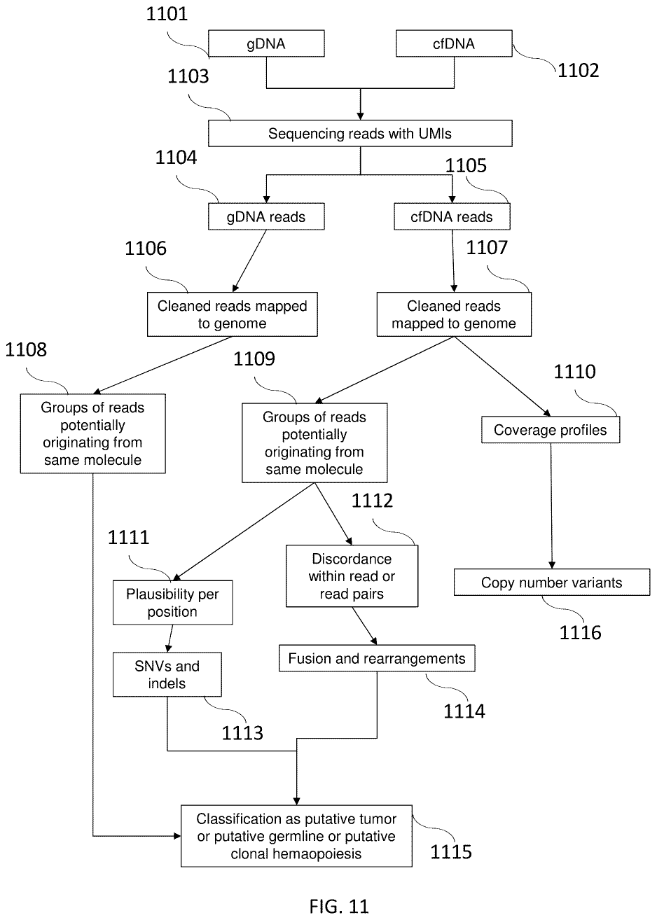

In embodiments where paired gDNA and cfDNA are processed jointly, different analyses may be conducted on reads originating from the gDNA versus read originating from the cfDNA sample. As illustrated in FIG. 11, gDNA 1101 and/or cfDNA 1102 are sequenced according to the methods described herein to produce sequencing reads with unique molecular identifiers 1103, which can be demultiplexed to separate gDNA reads 1104 and cfDNA reads 1105. In some embodiments, the cleaned cfDNA reads mapped to the genome 1107 and grouped into read groups, and discordance within reads or among pairs of reads 1112 are used to infer structural variants such as gene fusions and rearrangements 1114.

### Detection of Copy Number Variants

In some embodiments, read coverage may be computed from the aligned cleaned reads. Read coverage may be computed per pre-defined genomic region or among bins or a pre-defined length. Read coverage may then be normalized among regions and among samples. The normalization may use successive sets of other samples from the same batch as reference. In an iterative process, copy number variants (CNVs) may be estimated, and a set of samples with a similar coverage profile accounting for the CNV may be identified. Coverages may then be normalized based on the set of reference sample.

The process may be conducted iteratively, each time potentially using a different set of reference samples, until convergence is reached, a loop is produced, or a pre-defined maximum number of iterations is reached (WO2017/085243). Normalized read coverage may then be analyzed to identify statistical departures from the expected coverage indicative of copy number variants.

In embodiments where the whole-genome sequencing library is mixed with the capture library prior to sequencing, the coverage analyses may focus on either the target regions or the low-coverage whole-genome sequence data. As illustrated in FIG. 11, in embodiments where paired gDNA sample 1101 and cfDNA sample 1102 are analyzed, the analyses of coverage profiles 1110 may focus on reads originating from the cfDNA sample. In such embodiments, the inference of copy number variants 1116 may be based solely on cfDNA cleaned reads mapped to genome 1107.

### Identification of Short Genetic Variants

As illustrated in FIG. 10, a nucleic acid 1001, which can be cfDNA, gDNA, or cDNA, is sequenced according to the methods described herein to produce sequencing reads with unique molecular identifiers 1010. The sequencing reads 1010 can be pre-processed and mapped to a reference genome, producing cleaned reads mapped to the genome 1020. As described herein, the pre-processing may generate numerical codes for the space length adapters, which together with the mapping start-end positions, may be used to group reads into groups of reads potentially originating from the same molecule 1030. Using the methods described herein, the plausibility 1050 for each possible state may be calculated for each position, taking into account the read group information. The plausibility 1050 may then be used to identify SNVs and indels 1080.

As illustrated in FIG. 11, in some embodiments, paired gDNA 1101 and cfDNA 1102 from the same sample are sequenced according to the methods described herein to produce sequencing reads with unique molecular identifiers 1103, which can be demultiplexed to separate gDNA reads 1104 and cfDNA reads 1105. In some embodiments, the cfDNA reads 1107 are mapped to a reference genome 1107. In some embodiments, the gDNA reads 1104 are cleaned and mapped to a reference genome 1106. In some embodiments, groups of reads potentially originating from the same molecule 1108-1109 are determined from the cleaned and mapped sequencing reads 1107. In some embodiments, plausibility per position 1111 are calculated for the groups of reads potentially originating from the same molecule 1109 and SNVs and insertions/deletions (indels) 1113 are determined. In some embodiments, variant including SNVs and indels 1113 and fusion and rearrangements 1114 are classified as putative tumor, putative germline, or putative clonal hematopoiesis 1115, based on a comparison of the cfDNA groups of reads 1112 and gDNA groups of reads 1108.

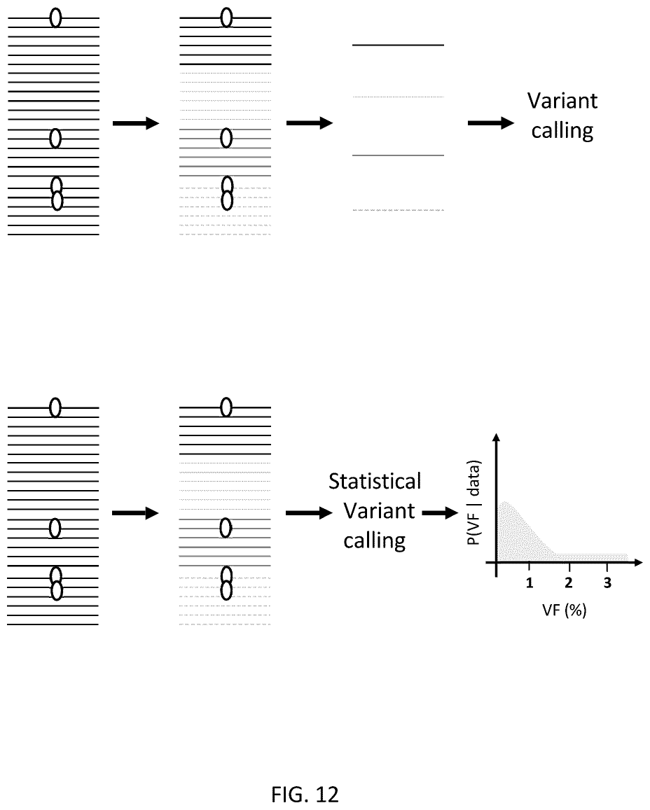

The resulting aligned reads may then be analyzed 353 to identify variants relative to the reference genome, such as SNVs, indels or structural variants (copy number variations, duplications, translocations . . . ). As illustrated by FIG. 12 and as reviewed for instance by Xu in “A review of somatic single nucleotide variant calling algorithms for next-generation sequencing data”, Computational and Structural Biotechnology Journal 16, pp. 15-24, February 2018, different approaches may be applied by the Genomic Data Analyzer 350. FIG. 12 illustrates the consensus sequencing approach, in which a single polynucleotide sequence is collapsed out of each group of sequence reads sharing, in the aligned BAM file, the same alignment position and numerical code tag according to the proposed method. If group members disagree at certain positions, as represented by circles in FIG. 12, various rules may be used to generate the consensus sequence which is then stored in a consensus BAM file (also known as read collapsed BAM file) as a single consensus aligned sequence read for each group of reads (family of reads corresponding to a parent fragment). The most frequently found base within the group may be kept as the consensus (simple majority rule). Quality scores may also be used to refine the consensus (weighted scoring). The resulting consensus sequence may then be processed by any conventional raw-reads-based variant callers. More generally, as will be apparent to those skilled in the art of NGS bioinformatics workflows, any consensus sequencing approach that is suitable as an intermediate step for collapsing the aligned reads into a single polynucleotide sequence prior to variant calling 353 may be used in combination with the proposed numerical code tag, similar to the processing of the UMI tag as in the public domain prior art methods reviewed by Xu, for instance with the MAGERI bioinformatics workflow (“MAGERI: Computational pipeline for molecular-barcoded targeted resequencing”, Shugay et al., PLOS Comput. Biol. 2017 May; 13(5)), or in various commercial genomic data analysis workflows, for instance with the Illumina Read Collapsing step (https://support.illumina.com/help/BaseSpace_App_UMI_Error_Correction_OLH_100000003 5906/Content/Source/Informatics/Apps/Read_Collapsing_appUMI.htm).

The above conventional consensus sequencing approaches however suffer from a number of limitations, which may be overcome by using more advanced genomic data analysis workflows based on advanced statistical modeling, such as data-driven methods derived from signal processing, or machine learning algorithms. FIG. 12 illustrates probabilistic sequencing as an alternative embodiment to the consensus sequencing approach. In probabilistic sequencing, instead of producing a consensus BAM file at an intermediate step between alignment and variant calling, the Genomic Data Analyzer 350 may directly use the raw fragment-tagged alignment file to feed the raw groups of aligned reads as input to a statistical variant caller.

Instead of relying on consensus sequences obtained with heuristic rules (such as, e.g., a majority vote), this class of variant callers relies on statistical models describing how instrumental artefacts affects reads belonging to the same or to different families (or groups). The statistical model can for example incorporate the knowledge that:
in the presence of a mutated DNA molecule, the variant is supported by all reads issued from the two strands of that mutated molecule;
sequencing errors can occur frequently, but independently across reads belonging or not to the same family;
PCR-errors are less frequent, but can affect multiple reads in the same family and rarely occur on both plus and minus strand of the same DNA molecule.

Analyzing the totality of reads within such a probabilistic framework allows, e.g., to compute the posterior probability of a variant allele frequency of interest. This posterior probability could then be used to, e.g., produce a variant call (e.g., if the probability of variant allele frequency>0 with probability p>threshold) and quantify its confidence level (i.e., the probability that the signal was generated by a real variant, rather than by instrumental noise).

One recently disclosed example of such a statistical variant caller is the SmCounter2 public domain stand-alone statistical variant caller which takes as input the aligned reads to calculate the variant probability in accordance with an error model based on a Beta distribution for the background error rates and a Beta-binomial distribution for the number of non-reference UMI outliers (“smCounter 2: an accurate low-frequency variant caller for targeted sequencing data with unique molecular identifiers”, Xu et al., Bioinformatics, Vol, 35(8), April 2019). As will be apparent to those skilled in the art of bioinformatics, the variant caller of SmCounter2 takes as input a BAM file including a duplex sequencing UMI tag for grouping reads issued from the same fragments. In the proposed workflow, instead of the UMI tag the BAM file may similarly include the numerical code tag of our proposed method, that is the pair of numerical values corresponding to the measured lengths of the variable adaptors ligated on each end of the ligated fragment according to the proposed wet lab method. Similar to SmCounter2, various variant callers from commercial workflows based on data-driven modeling, such as for instance the SOPHIA GENETICS Data-Driven Medicine software (SOPHIA DDM) may also be adapted to individually call variants for each group of aligned reads issued from different DNA fragments based on the proposed numerical code tagging.

A probabilistic method to identify SNVs and indels from cleaned and mapped reads that takes into account the UMIs information is disclosed here. This method integrates the full breadth of information from the reads, provided benefits in the presence of various sources of experimental noises.

Sequencing reads potentially originating from the same DNA molecule are identified based on the combination of the length of their molecular barcodes, recorded during the cleaning of reads as numerical codes, and the start and end positions of the sequences mapped to the genome. The same tag is added to all reads potentially originating from the same DNA molecule. Reads with different tags are considered as originating from different DNA molecules in the original sample. Reads with the same tag may originate from the same DNA molecule in the original sample.

Short genetic variants including single-nucleotide variants (SNVs) and insertion-deletions (indels) are identified based on the aligned trimmed reads, using a probabilistic variant calling approach. As disclosed below, the plausibility of different states (e.g. different nucleotides, groups of nucleotides, or indels) is calculated per position. The plausibility observed within groups of reads potentially originating from the same original molecule (read groups) is aggregated among read groups and used for variant calling, alongside other sources of information. The approach thereby incorporates metrics for the strength of the signal observed both within and across the read groups. In some embodiments, the approach further incorporates information about the concordance between forward and reverse reads, the concordance between reads originating from each strand in the original molecules (Watson and Crick strands), or the levels of noise expected in the absence of true variants.

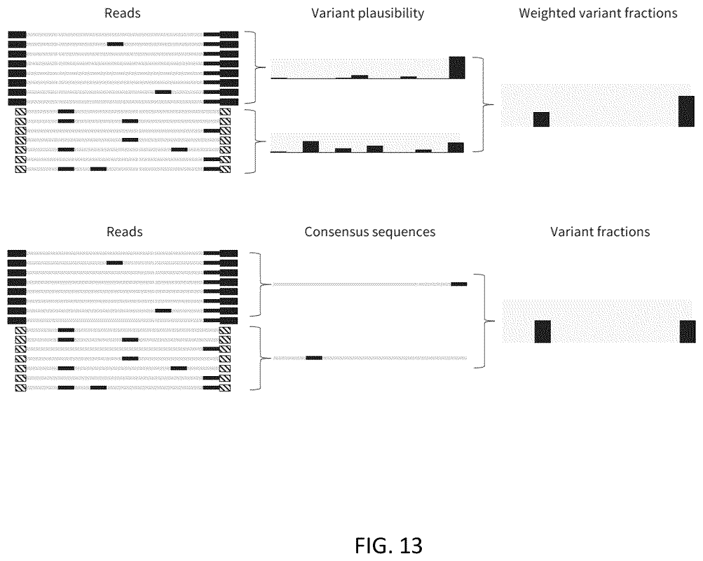

FIG. 13 provides a high-level comparison of the invention disclosed here at the top and the standard consensus approach at the bottom. Aligned reads are illustrated with grey bars on the left. The rectangles on the extremities represent UMIs, with shared colors indicating the reads share the same tag and therefore belong to the same read group. Black segments represent variants supported by the read. In the consensus method, a single sequence is inferred based on each read group, and the variant fraction is then obtained by counting the number of consensus sequences supporting the variant out of the total number of consensus sequences. In the method disclosed here, the read-group information is used to compute the plausibility of a variant being present at each position. An aggregated plausibility is then inferred across read groups, and can be used to calculate support with different metrics, or weighted variant fractions based on the plausibility.

For illustration purposes, the methods is detailed below as a two-step process, calculating for each position first the plausibility of states for each read group, and then aggregating the read-group plausibilities among all read groups. The method can however be implemented as a single step, with direct calculation of the aggregated plausibility taking into account the read-group information. It will be apparent to those skilled in the art that a single-step implementation allows for modification and optimization of parameters and therefore may offer computational advantages.

### Plausibility of States Per Group of Reads

For each genomic i among the regions targeted by the capture probes, all sequencing reads spanning the position are considered. A plausibility plaus^{N}ᵢⱼ of a given state N existing at position i is calculated for each group of reads potentially originating from the same molecule j. The plausibility plaus^{N}ᵢⱼ can take into account any combination factors that include among others the number of reads supporting the state N, the sequencing quality at the position, the quality across the reads, the distribution of the signal among forward and reverse reads, the distribution of the signal among the reads originating from each strand in the original molecule (Watson and Crick strands), the nucleotide diversity among phased alleles, the co-occurrence of softclips, or other sources of information.

In some embodiments, the states N considered as possible for a given position are the four nucleotides adenine (A), cytosine (C), guanine (G), and Thymine (T). In some embodiments, uracil (U) represents an additional possible state. In some embodiments, modified bases, for example through methylation, constitute additional possible states. In some embodiments, an additional state N representing a deletion compared to the reference genome is considered as possible. In some embodiments, any length of base pair deletion compared to the reference genome is considered as a possible state, each length representing a different state N. In such embodiments, the deletion may be assigned to the left- or right-anchor position independently of the deletion length. The deletion would therefore be counted as a supported state only for the anchor position. In some embodiments, any number of base pair insertions and any identity of inserted nucleotides compared to the reference genome is considered as possible states N, and each length might be considered as a different state. In such embodiments, the insertion may be assigned to the left- or right-anchor position independently of the insertion length. In such embodiment, insertion of different bases at the same position (e.g. ‘AA’ vs ‘CT’) may be considered as different states N.

In some embodiments, the plausibility plaus^{N}ᵢⱼ of the state N being present at position i in read group j is inferred based on the number of reads supporting each state at position i within read group j. In some embodiments, the number of reads nreads^{N}ᵢⱼ supporting each of the states N observed at position i among the read groups j is recorded. In some embodiments, the state N1 supported by highest number of reads at position j is identified, and the state N2 supported by the second highest number of reads at position j is identified. The plausibility plaus^{N}ᵢⱼ of the state N1 at position j may then be calculated as the difference between nreads^{N1}ᵢⱼ and nreads^{N2}ᵢⱼ, so that plaus^{N1}ᵢⱼ=nreads^{N1}ᵢⱼ−nreads^{N2}ᵢⱼ. In some embodiments, this difference might be divided by nreads^{N1}ᵢⱼ or by the total number of reads at position j, providing values comprised between 1 (all reads support the same nucleotide) and 0 (same number of reads for N1 and N2 states), wherein plaus^{N1}ᵢⱼ=(nreads^{N1}ᵢⱼ−nreads^{N2}ᵢⱼ)/nreads^{N1}ᵢⱼ or plaus^{N1}ᵢⱼ=(nreads^{N1}ᵢⱼ−nreads^{N2}ᵢⱼ)/Σnreads^{N}ᵢⱼ, wherein Σnreads^{N}ᵢⱼ is the total number of reads from read group j covering position i. In some embodiments, plaus^{N1}ᵢⱼ may be calculated as the difference between the number of reads nreads^{N1}ᵢⱼ supporting the state N1 with the highest support and the number of reads nreads^{N3}ᵢⱼ supporting the state N3 with the third highest support, with the difference being divided for example by the number of reads supporting N1, such that plaus^{N1}ᵢⱼ=(nreads^{N1}ᵢⱼ−nreads^{N3}ᵢⱼ)/nreads^{N1}ᵢⱼ. In such embodiments, the plausibility plaus^{N2}ᵢⱼ for the state with the second highest support may then be calculated as plaus^{N2}ᵢⱼ=(nreads^{N2}ᵢⱼ−nreads^{N3}ᵢⱼ)/nreads^{N1}ᵢⱼ. It will be apparent to those skilled in the art that the approach can be extended to more states and that different mathematical relationships, such as a ratio between nreads^{N1}ᵢⱼ and nreads^{N2}ᵢⱼ, or the like, may be used.

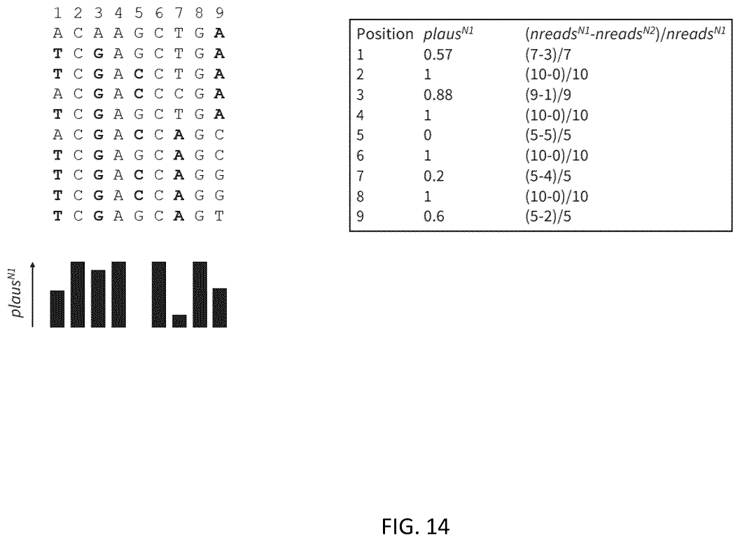

FIG. 14 provides an illustration of an embodiment where the plausibility plaus^{N1} is inferred based on the number of reads covering position i in the read group j. Ten different reads belonging to the same read group, wherein each read is 9 base pair long, are illustrated on the left, with positions aligned vertically and numbered at the top. The detailed calculations are illustrated with one possible embodiment on the right. Positions 2, 4, 6 and 8, are covered by reads that all support the same state and the state is therefore assigned a plausibility of 1. Position 5 is covered by the same number of reads harboring each of two different states and therefore both states receive a plausibility of 0. The plausibility of the state at positions 1, 3, 7 and 9 depends on the number of reads supporting each state. As exemplified by position 7, the plausibility is minimal if the second state N2 receives a level of support that is close to the level of support of the first state N1. In this example, the first state N1 at positions 5, 7 and 9 is supported in each case by five reads. The plausibility increases at position 7 and further at position 9 because support for the second state is each time lower.

In some embodiments, the plausibility plaus^{N}ᵢⱼ takes into account the sequencing quality at each position i and for each read within the read group j. The overall quality qual^{N}ᵢⱼ may be calculated for each state N. In some embodiments, the state quality qual^{N}ᵢⱼ may be calculated as the sum of the individual sequencing quality scores (e.g. phred scores) at position i for all reads from the read group j supporting the state N. In some embodiments, the state quality qual^{N}ᵢⱼ may be calculated as the sum of the individual sequencing quality scores (e.g. phred scores) at position i and the positions on each side for all reads from the read group j supporting the state N. In some embodiments, the state quality qual^{N}ᵢⱼ may be calculated as the sum of the individual sequencing quality scores (e.g. phred scores) at position i and the positions over a window of a given length of base pairs, where the window length may be any number up to the read length, centered on position i for all reads from the read group j supporting the state N. Different mathematical operations, and for each read, the average of median of the score at position i and at adjacent positions might be used. In some embodiments, the state quality qual^{N}ᵢⱼ may be calculated as the sum of read-level quality metrics for all reads from the read group j supporting the state N at position i. The plausibility plaus^{N}ᵢⱼ may be calculated by comparing the state quality qual^{N1}ᵢⱼ of the state N1 having the highest state quality to the state quality qual^{N2}ᵢⱼ of the state N2 having the second highest state quality. In such embodiments, the difference between the two may be normalized by dividing by qual^{N1}ᵢⱼ, such that plaus^{N1}ᵢⱼ=(qual^{N1}ᵢⱼ−qual^{N2}ᵢⱼ)/qual^{N1}ᵢⱼ. In some embodiments, the plausibility plaus^{N}ᵢⱼ may be calculated by comparing the state quality qual^{N1}ᵢⱼ of the state N1 having the highest state quality to the state quality qual^{N3}ᵢⱼ of the state N3 having the third highest state quality score, for example as plaus^{N1}ᵢⱼ=(qual^{N1}ᵢⱼ−qual^{N3}ᵢⱼ)/qual^{N1}ᵢⱼ. In such embodiments, the plausibility of N2 may then be calculated as plaus^{N2}ᵢⱼ=(qual^{N2}ᵢⱼ−qual^{N3}ᵢⱼ)/qual^{N1}ᵢⱼ. It will be apparent to those skilled in the art that the approach can be extended to more states and that other mathematical relationships are possible.

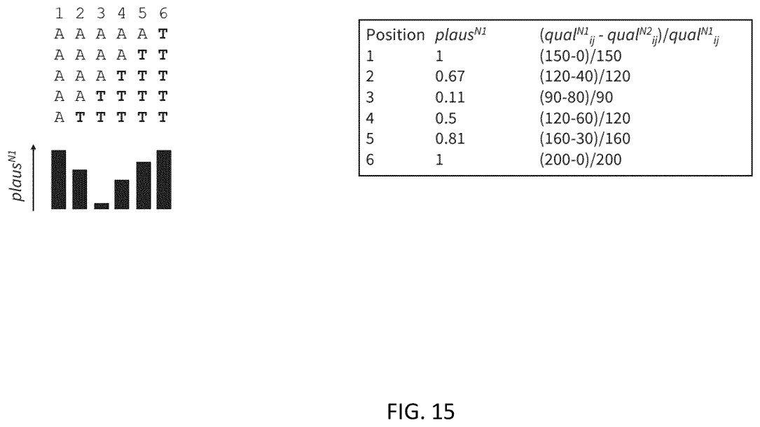

An example of plausibility calculations based on quality scores is provided in FIG. 15. In this simplified scenario, five sequences, each composed of six nucleotides, are shown on the left, with positions aligned vertically and numbered at the top. For simplicity purposes, all As are assigned a quality score of 30, while all Ts are assigned a quality score of 40. The calculations of plaus^{N1} with this embodiment are detailed on the right. The resultant plausibility plaus^{N1} are indicated below with a bar chart. It is apparent that both the number of reads and the sequencing quality affect the calculated plaus^{N1}, as the score is higher at position 4 than at position 3, despite the number of reads supporting the most frequent nucleotide being equal. It will be apparent to those skilled in the art that, in practice, the quality score can vary among reads supporting the same state.

In some embodiments, the plausibility plaus^{N}ᵢⱼ might further take into account both the quality of the reads supporting the state N at position i within the read group j, and the number of such reads. As a non-limiting example, plaus^{N}ᵢⱼ for state N1 might be calculated as plaus^{N1}ᵢⱼ=nreads^{N1}ᵢⱼ*(qual^{N1}ᵢⱼ−qual^{N2}ᵢⱼ)/qual^{N1}ᵢⱼ, wherein Σnreads^{N}ᵢⱼ is the number of reads from read group j covering position i. As another non-limiting example, plaus^{N}ᵢⱼ for state N1 might be calculated as plaus^{N1}ᵢⱼ=nreads^{N1}ᵢⱼ/Σnreads^{N}ᵢⱼ*(qual^{N1}ᵢⱼ−qual^{N2}ᵢⱼ)/qual^{N1}ᵢⱼ, wherein Σnreads^{N}ᵢⱼ is the number of reads from read group j covering position i. It will be apparent to those skilled in the art that other mathematical formulas and transformations are possible.

In some embodiments, the quality qual^{N}ᵢⱼ may be modified in function of the nature of the state N. As an example, the quality of reads supporting states corresponding to insertions or deletions might be decreased by a given factor to penalize the inference of indels. The value of the penalty might further increase with the length of the indel. As a non-limiting example, a factor corresponding to 1/(1+indel_length), wherein indel_length represents the number of bases inferred to be deleted, might be used to multiply the quality of each read at position i. As another example, the quality of reads supporting states that frequently result from mutations in some storage conditions might be lowered by a given factor. As a further example, the quality of reads supporting states that differ from the reference genome or that are not observed in reference populations might be decreased by a given factor. It will be apparent to those skilled in the art that such corrections and others allow to penalize mutations that are known to frequently represent artifacts, and can be implemented through other mathematical transformations.

In some embodiments, the quality qual^{N}ᵢⱼ may be modified as a function of the quality of the alignment of the reads. As a non-limiting example, qual^{N}ᵢⱼ may be multiplied by a factor inversely proportional to the number of reads supporting the state N at position i that present a portion at the extremity that does not align to the reference genome (soft-clip). As another non-limiting example, qual^{N}ᵢⱼ may be multiplied by a factor representing the length of the reads supporting state N at position i in group j that is aligned to the reference genome, thereby lowering the quality if only portions of reads are aligned to the region surrounding i.

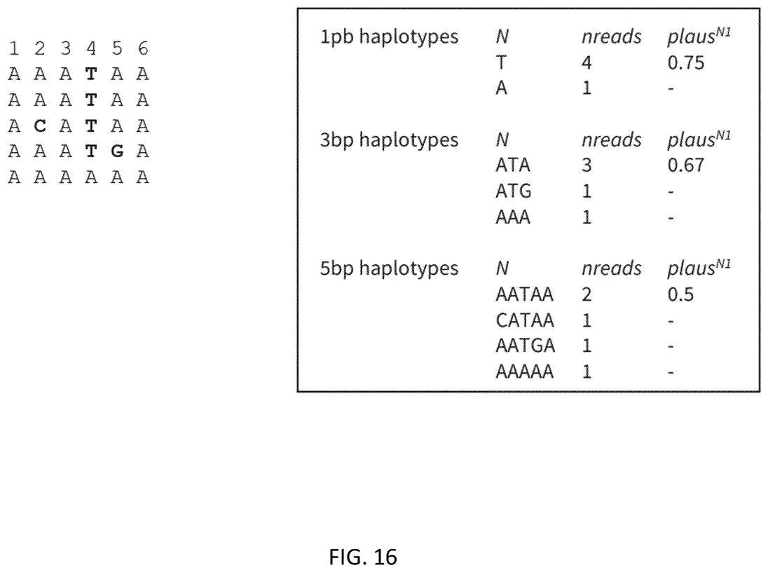

In some embodiments, the plausibility plaus^{N}ᵢⱼ is calculated for each state N observed at position i in read group j, where the states N correspond to haplotypes (nucleotides from the same read) of a given length, where the length can be any number up to the read length. For each of the N states, the plausibility can then be inferred by any of the methods illustrated above. It will be apparent to those skilled in the art that calculating the plausibility per haplotype has the effect of decreasing the plausibility when a diversity of nucleotides is observed on adjacent positions. FIG. 16 provides an illustration of the concept for five sequences of six base pairs each, with positions aligned vertically and numbered at the top. The number of states observed at position 4 are listed on the right, focusing on 1 bp haplotypes (single nucleotide, two states N), 3 bp haplotypes (three states N in this example) or 5 bp haplotypes (five states N in this example) centered on position 4. The resultant number of read and plausibility are indicated, using the embodiment where plaus^{N1}ᵢⱼ=(nreads^{N1}ᵢⱼ−nreads^{N2}ᵢⱼ)/nreads^{N1}ᵢⱼ. This example is for illustration purposes only and other corrections can be integrated possible, including for the sequencing quality or absolute numbers of reads, as disclosed above.

In some embodiments, the plausibility calculations take into account the concordance between forward and reverse reads or between reads originating from each of the two strands (Watson and Crick strands) in the original molecules. In some embodiments, a concordance score concᵢⱼ may be inferred among subgroups of reads, corresponding to forward reads versus reverse reads, from each read group j covering position i. In some embodiments, a concordance score concᵢⱼ may be inferred among subgroups of reads, corresponding to reads originating from the strand in the same direction as the reference genome versus reads originating from the complementary strand, from each read group j covering position i. As a non-limiting example, the concordance score might be extracted from a contingency table reporting the number of occurrences of the observed states among each of the forward versus reverse reads or the two original strands, and the concordance score might be derived from any of different statistics, such as those involved in Fisher's exact test or chi-square tests, potentially with transformation of the statistics to impose a desired penalty on states receiving different support among the types of reads. In an embodiment where the calculated concᵢⱼ ranges from 0 (completely different information among the types of reads) to 1 (complete concordance), the plausibility plaus^{N1}ᵢⱼ might then be calculated as plaus^{N1}ᵢⱼ=concᵢⱼ*(qual^{N1}ᵢⱼ−qual^{N2}ᵢⱼ)/qual^{N1}ᵢⱼ. Other transformations may be included as disclosed above. The concordance score concᵢⱼ might be calculated among all states observed at position i in read group j or among specific states.

In some embodiments, the plausibility may be calculated independently for two subgroups within each read group j, which might correspond to forward versus reverse reads or to reads originating from each of the two strands (Watson and Crick strands) in the original molecule. As a non-limiting example, the plausibility of the forward reads Fplaus^{N1}ᵢⱼ might be calculated as Fplaus^{N1}ᵢⱼ=(Fqual^{N1}ᵢⱼ−Fqual^{N2}ᵢⱼ)/Fqual^{N1}ᵢⱼ, wherein Fqual^{N1}ᵢⱼ is the quality for position i summed among read of read group j calculated using one of the methods disclosed above considering only one subgroup of reads, and Rplaus^{N1}ᵢⱼ might be calculated similarly considering only reads from the other subgroup. In such embodiments, the plausibility calculated from each subgroup of reads might be combined in a way that maximizes the final plausibility of the state N in read group j if high scores are obtained for both subgroups. As a non-limiting example, the product of the plausibility inferred from the two subgroups groups of reads might be calculated, so that plaus^{N1}ᵢⱼ=Fplaus^{N1}ij*Rplaus^{N1}ᵢⱼ. As another example, the root square of the product of the plausibility inferred from the two subgroups of reads might be calculated, so that plaus^{N1}ᵢⱼ=sqrt(Fplaus^{N1}ᵢⱼ*Rplaus^{N1}ᵢⱼ). As another example, the smallest of the plausibility inferred for the two subgroups might be inferred, so that plaus^{N1}ᵢⱼ=min (Fplaus^{N1}ᵢⱼ, Rplaus^{N1}ᵢⱼ) It will be apparent to those skilled in the art that different mathematical formulas can be used.

In some embodiments, an adjusted coverage adjCov^{N}ᵢⱼ is calculated for each position i, for each read group j, by multiplying the coverage, in number of reads nreads^{N}ᵢⱼ, by the plausibility plaus^{N}ᵢⱼ, so that adjCov^{N}ᵢⱼ=nreads^{N}ᵢⱼ*plaus^{N}ᵢⱼ. As a non-limiting example, the plausibility plaus^{N}ᵢⱼ, calculated for the state N1 based on the quality scores may be used, so that adjCov^{N1}ᵢⱼ=nreads^{N1}ᵢⱼ*(qual^{N1}ᵢⱼ−qual^{N2}ᵢⱼ)/qual^{N1}ᵢⱼ. It will be apparent to those skilled in the art that any of the plausibility metrics disclosed here may be used and that different mathematical formulas can produce similar effects. In some embodiments, adjCov^{N}ᵢⱼ may be calculated for each state N. In some embodiments, adjCov^{N}ᵢⱼ may be calculated only for the state N1 with the highest plausibility.

### Aggregated Support Metrics

Support metrics for each state N at each position i can be calculated from the plausibility plaus^{N}ᵢⱼ for the different read groups j. In some embodiments, for each state N with the highest plausibility in at least one of the j read groups, the weighted number of groups supporting the state N at position i wngroup^{N}ⱼ is obtained by summing plaus^{N}ᵢⱼ, across all read groups j where the state N is the state with the highest plausibility, from 1 to n, wherein n is the total number of groups:

$${wngroup}_{i}^{N} = {\sum }_{j = 1}^{n} {plaus}_{ij}^{N = N 1}$$

It will be apparent to those skilled in the art that this approach counts differently read groups with high support for the state from read groups with moderate support for the state. In some embodiments, the weighted number of groups wngroup^{N}ⱼ is obtained from all read groups j where the state N is either the state N with the highest plausibility (in which case plaus^{N1}ᵢⱼ is considered) or the state N2 with the second highest plausibility (in which case plaus^{N2}ᵢⱼ is considered). In some embodiments, only read groups where plaus^{N1}ᵢⱼ (or plaus^{N2}ᵢⱼ) is above a given threshold are included. In some embodiments, different support metrics are obtained, for example by independently calculating the sum of plaus^{N1}ᵢⱼ inferred from read numbers and plaus^{N1}ᵢⱼ inferred from sequencing quality scores, using the methods disclosed above.

In some embodiments, a support metric representing the weighted number of duplex groups supporting the state N at position i wndup^{N}ᵢ is obtained using the plausibility plaus^{N1}ᵢⱼ, obtained by combining the plausibility Fplaus^{N1}ᵢⱼ calculated based on reads originating from the strand in the same orientation as the reference genome and the plausibility Rplaus^{N1}ᵢⱼ based on reads originating from the strand in reverse complement compared to the reference genome. The two plausibility Fplaus^{N1}ᵢⱼ and Rplaus^{N1}ᵢⱼ can be combined using the product, the square root of the product, the lowest of the two, or another method. As a non-limiting example, the weighted number of duplexes supporting the state N at position i wndupl^{N}ᵢ might be calculated as the sum, across all groups j between 1 and n, where n is the total number of read groups:

$${wndupl}_{i}^{N} = {\sum }_{j = 1}^{n} min ( {Fplaus}_{ij}^{N = N 1} , {Rplaus}_{ij}^{N = N 1} )$$

In some embodiments, the weighted number of duplex groups wndup^{N}ᵢ is obtained using the same method, but considering only the read groups where both original strands are covered by a number of reads exceeding a given threshold. In some embodiments, the weighted number of duplex groups wndupl^{N}ᵢ is obtained using the same method, but considering only the read groups j for which the concordance concᵢⱼ among subgroups originating from different strands in the original molecule exceeds a given threshold. In some embodiments, a weighted number of simplexes is calculated by aggregating the sum of Fplaus^{N1}ᵢⱼ and Rplaus^{N1}ᵢⱼ for read groups where either Fplaus^{N1}ᵢⱼ or Fplaus^{N1}ᵢⱼ is equal to 0.

In some embodiments, a weighted read coverage wnread^{N}ᵢ is obtained per state N per position i by summing all the adjusted coverage adjCov^{N}ᵢⱼ across read groups j. In some embodiments, the weighted read coverage is obtained per state N per position i by summing all the adjusted coverage adjCov^{N}ᵢⱼ across read groups j, considering only the read groups where N is the state with the highest plausibility. As a non-limiting example, the weighted read coverage wnread^{N}ᵢ for state N at position i can be obtained across all groups between 1 and n, where n is the total of groups, as:

$${wnread}_{i}^{N} = {\sum }_{j = 1}^{n} {adjCov}_{ij}^{N = N 1}$$

In some embodiments, a weighted variant allele fraction is obtained based on the inferred support for a given state N at position i across all read groups j. For example, the variant allele fraction for state N can be computed as the sum of plaus^{N1}ᵢⱼ, for all read groups j where state N has the highest plausibility, divided by the sum of all plaus^{N1}ᵢⱼ independently of the nature of N1. It will be apparent to those skilled in the art that this is equivalent to calculating wngroup^{N=N1}ⱼ, divided by the sum of wngroup^{N}ⱼ for all states. This weighted group variant allele fraction for state N at position i wgvaf^{N}ᵢ, can thus be expressed as:

$${wgvaf}_{i}^{N 1} = {wngroup}_{i}^{N 1} / {\sum }_{N = 1} {wngroup}_{i}^{N}$$

In some embodiments, a weighted variant allele fraction is obtained based on the inferred support for a given state N at position i across all read groups j in weighted number of duplexes wdupl^{N}. In such embodiments, the weighted duplex variant allele fraction wdvaf^{N}ᵢ may be calculated as wndupl^{N=N1}; divided by the sum of wndupl^{N}ⱼ for all states N:

$${wdvaf}_{i}^{N 1} = {wndupl}_{i}^{N 1} / {\sum }_{N = 1} {wndupl}_{i}^{N}$$

In some embodiments, a weighted variant allele fraction is obtained based on the inferred support for a given state N at position i across all read groups j in weighted number of reads wnread^{N}ᵢ. In such embodiment, the weighted read variant allele fraction wrvaf^{N}ᵢ may be calculated as wnread^{N=N}ⱼ, divided by the sum of wnread^{N}ⱼ for all states N:

$${wrvaf}_{i}^{N 1} = {wnread}_{i}^{N 1} / {\sum }_{N = 1} {wnread}_{i}^{N}$$

It will be apparent to those skilled in the art that the aggregated support metrics presented here can be calculated in a single operation from all reads covering position i, taking into account the read group to which they belong.

In some embodiments, the number of reads supporting a variant expected at a position in the absence of real variant (background noise) is calculated based on sequence data from previously sequenced samples, which may be reference samples, uncharacterized samples, or a combination thereof. In some embodiments, the signal expected at position i in the absence of variant may be calculated using the plausibility and aggregated support metrics disclosed here. As a non-limiting example, the expected support metric may be calculated based on sequencing data, potentially obtained previously, corresponding to samples not expected to harbor a sample at variant i. In some embodiments, the number of reads supporting a variant expected at a position in the absence of real variant is calculated based on the same sequence data from which the variants are being detected. As a non-limiting example, the background noise may be computed per position i as metrics capturing the diversity of nucleotides at positions adjacent to i or located within a window of pre-defined size adjacent to position i or overlapping position i. As another non-limiting example, the background noise may be computed per position i as metrics capturing the diversity of haplotypes observed within a window of pre-defined size adjacent to position i or overlapping position i. The background noise, obtained with any of the mentioned methods, may be transformed to correspond to the probability bgnᵢ of a read at position i supporting a variant in the absence of said variant in the original sample.

In some embodiments, a score might be calculated to assess the likelihood of observing the distribution of the support for a given state among different categories of reads. As a non-limiting example, the support, in weighted read coverage, for the states N corresponding to the reference and alternative variant might be calculated, for the forward and reverse reads, where Fwnreads^{ref}ᵢ, is the weighted read coverage based on forward reads for the reference allele at position i, Rwnreads^{ref}ᵢ is the weighted read coverage based on reverse reads for the reference allele at position i, and Fwnreads^{alt}ᵢ and Rwnreads^{alt}ᵢ are the same support metrics for the alternative allele. A score representing the compatibility of the observed data with expected proportionality of support among forward and reverse reads may then be calculated, for example using a statistic on the contingency table given by ((Fwnreads^{alt}ᵢ, Rwnreads^{alt}ᵢ), (Fwnreads^{ref}ᵢ, Rwnreads^{ref}ᵢ)). In some embodiments, the score may be the p-value or odds ratio from a Fisher Test, the p-value or the Chi-square of a Chi-square test, or a statistic from another test, potentially with some numerical transformation. In some embodiments, the score may be calculated using the same method, but by separating reads among those originating from each of the two strands (Watson and Crick) in the original molecule. In some embodiments, the score may be calculated by comparing the observed weighted read coverage wnreads^{ref}ᵢ and wnreads^{alt}ᵢ to the weighted coverage for the reference and alternative alleles expected at position i in the absence of variant, wherein the expected weighted coverages in the absence of variant are based on calculations of background noise. In such embodiments, the contingency table may be given as ((wnreads^{ref}ᵢ, wnreads^{alt}ᵢ), ((1−bgnᵢ)*(wnreads^{ref}ᵢ+wnreads^{alt}ᵢ), bgnᵢ*(wnreads^{ref}ᵢ+wnreads^{alt}ᵢ))), wherein bgnᵢ is the rate of false support expected at position i in the absence of variants. It will be apparent to those skilled in the art that the score disclosed here may be calculated using the weighted number of groups wngroup^{N}ᵢ or weighted number of duplexes wndupl^{N}ᵢ instead of the weighted read coverage. In some embodiments, different scores may be combined using mathematical relationships to obtain a composite score capturing the likelihood of the patterns. The combination may include summing, with different weighting factors, multiplying, or taking the lowest score.

### Variant Calling

The different support metrics and noise metrics disclosed here can be integrated to determine whether a given state N at each position i is likely to represent a variant present in the original sample as opposed to an artifact originating during the library preparation or the sequencing. In some embodiments, variants are called if one or several support metrics are above a pre-defined threshold. In some embodiments, variants are called if the weighted number of groups wngroup^{N}ᵢ supporting the variant is above a pre-defined threshold. In some embodiments, variants are called if the weighted number of duplexes wngroup^{N}ᵢ supporting the variant is above a given threshold. In some embodiments, variants are called if the weighted read coverage wnread^{N}ᵢ is above a given threshold. In some embodiments, variants are called if the weighted variant allele fraction is above a pre-defined threshold, wherein the variant allele fraction may be any of wgvaf^{N}ᵢ, wdvaf^{N}ᵢ, or wrvaf^{N}ᵢ. In some embodiments, the variant is called if a rule that is based on any combination of these metrics is fulfilled.

In some embodiments, variants are called if the considered support metrics statistically exceed those expected in the absence of variants, based on the background noise, using a pre-defined p-value threshold.

In some embodiments, variants are called if a score assessing the distribution of support among reads, obtained with the method disclosed here, exceeds a given threshold of deviation from the expected patterns. In some embodiments, variants are called if more than one score exceeds a given threshold of deviation from the expected patterns.

In some embodiments, the thresholds for variant calling are set up by experts to fit the target application. In some embodiments, the thresholds are optimized to maximize the number of true positives/negatives and minimize false positives/negatives, using data obtained for reference samples with known variants. It will be apparent to those in the arts that such optimization can use training and validation datasets and can involve different heuristics and optimization techniques known in the art. In some embodiments, the thresholds are selected to match those used in previous applications that are similar in some respect the target application. In some embodiments, the thresholds are optimized during the variant calling process to achieve a given proportion of variants. In some embodiments, the thresholds are optimized during the variant calling process to maximize the number of true positives and true negatives based on positions with known statuses in the target sample or based on a reference sample included in the same sequencing run. In some embodiments, the thresholds are optimized during the variant calling process to minimize the probability of observing the called variants by chance.

In some embodiments, a combination of different support metrics, p-values, and scores, are used to call variants. In some embodiments, variants are reported with different confidence levels depending on the number of metrics reaching the necessary thresholds. In some embodiments, the thresholds are lowered for variants previously detected in the sample subject. In some embodiments, the thresholds are lowered for variants previously detected in other subjects having a similar condition. In some embodiments, the thresholds are modified for variants previously detected in a reference population.

For each called variant, the support is reported with metrics that can include one or a combination of those disclosed here, as well as the total number of reads and the read allele fraction, calculated using methods known in the art. In some embodiments, support metrics calculated using a consensus-based approach known in the art might also be reported. In some embodiments, the variants are compared to databases of genetic variants, which can include variants previously detected in the same patient (internal database), variants previously detected in other subjects with the same type of condition (internal or external databases), variants previously detected in other subjects with other conditions (internal or external databases), variants previously detected in healthy subjects (internal or external databases), variants not previously detected. In such embodiments, variants may be reported in different categories depending on whether they were previously detected in the same subject, whether they were previously reported in other subjects with the same or similar condition, or whether they were not reported in the same subject, other subjects with the same condition, or other subjects with other conditions. In some embodiments, different support thresholds are used among these categories to report variants.

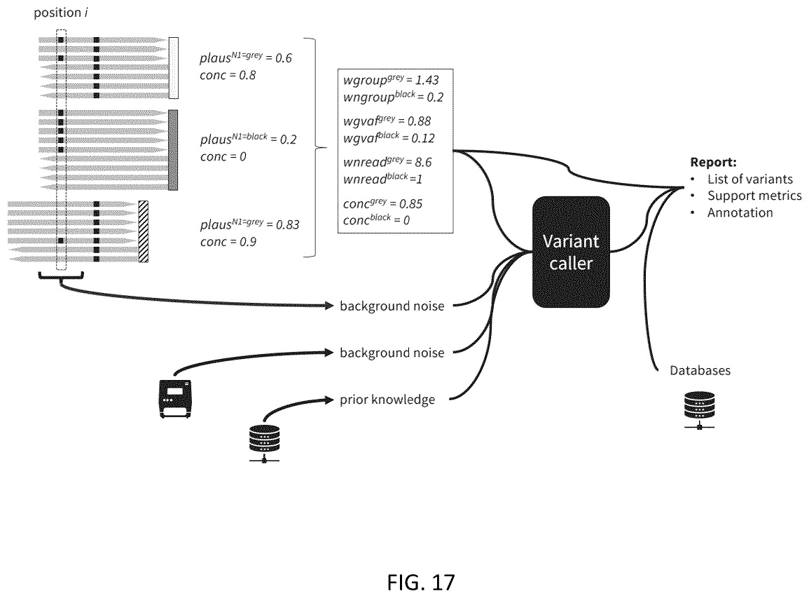

An illustration of one possible embodiment is provided in FIG. 17. Three read group are illustrated on the left, with bars on the side indicating they share the same start end positions and molecular barcodes. The arrows represent forward reads (right direction) versus reverse reads (left direction). The black points represent variants compared to a reference. The method considers each position i consecutively, with one highlighted with a box. Plausibility metrics are calculated per read group. The read-group level plausibilities are then used to calculate overall support metrics. In this example, hypothetical concordance metrics conc are calculated. The overall metrics are used, alongside the sequencing noise calculated from the same sequence data, the background noise calculated from reference sequencing data, and prior knowledge, such as the occurrence in a reference population, and used by a variant caller. The variant caller returns a list of variants, to which support metrics are associated. Annotation can be retrieved from databases, such as the frequency in reference populations, clinical association, predicted pathogenicity, and the like. This information can be included in a report.

### Identification of Large Structural Variants

In some embodiments, structural variants, such as gene fusions, intra-chromosomal rearrangements, or inter-chromosomal rearrangements may be detected by analyses of reads that include regions mapping to different parts of the genome, or pairs of reads where each reads maps to different parts of the genome.

In some embodiments, for each read with subparts mapping to different regions of the genome, the two likely regions of origin may be identified as those matching each of the subpart based on a read mapping or sequence matching tool. For each read with subparts mapping to different regions of the genome, the likely breakpoint between the two regions is identified based on the degree of divergence from the reference on the 5′ and 3′ sides, the most likely breakpoint being the position leading to most differences with the reference on one of the two sides. All reads with subparts corresponding to the same two likely regions of origin are considered jointly and the distribution of their likely breakpoint with respect to the reference genome is recorded. The distribution of the breakpoints with respect of each of the two likely regions of origin is used to establish the most likely breakpoint based on multivariate statistical modelling. A fusion involving two different genomic origins may be inferred if the supporting evidence exceeds a given threshold. In some embodiments, a fusion is reported if the number of reads, or groups of reads potentially originating from the same molecule, supporting the same breakpoint and the same two regions of origins exceeds a pre-defined threshold. In some embodiments, the threshold may differ based on whether the fusion was previously detected in the same sample, was previously detected in other samples with the same or similar cancers, or was not previously detected.

In some embodiments, for each pair of reads where each of the two reads map to a different genomic location, the two likely regions of origin may be recorded. In some embodiments, for each such pair of reads, the genomic region where the breakpoint may be located is recorded as the region starting from the end of each read and extending to the expected maximal DNA insert length, which can be set based on a pre-defined value, based on measured fragment length during the library preparation, or based on the insert length observed for pairs of sequencing reads from the same dataset where the two reads map to the same genomic region. Pairs of reads where the two reads map to the same two distinct genomic locations may be grouped. In some embodiments, the likely breakpoint may be inferred based on the overlap of possible breakpoints among these pairs of reads. The support for a fusion involving the two identified genomic regions may be recorded as the number of pairs of reads, or number of groups of pairs of reads potentially originating from the same molecule, with one read mapping onto each of the two regions. In some embodiments, a fusion event may be report if the support exceeds a pre-defined threshold. In some embodiments, the threshold may differ based on whether the fusion was previously detected in the same sample, was previously detected in other samples with the same or similar cancers, or was not previously detected.

### Distinction Between Somatic, Germline and Clonal Hematopoietic Variants

In embodiments where cfDNA and gDNA from the same patient are analyzed in parallel, the likely origin of detected variants may be inferred by comparing the allele frequency of detected variants between the cfDNA and gDNA sample, as illustrated in FIG. 11. The comparison may be made using the weighted variant allele fraction, but those skilled in the art will understand that other metrics may be used, including those disclosed above or those known in the art.

In some embodiments, the cleaned gDNA reads 1106 assigned to read groups 1108 are screened for all positions where a variant, such as a single nucleotide variant (SNV), a short insertion/deletion (indel), or a fusion was detected in the cfDNA reads 1107 assigned to read groups 1107. For all gDNA reads covering the genomic positions, the number of reads supporting the variant are reported. In addition, the support metrics based on aggregated plausibility of the variants among read groups are reported. In some embodiments, variants are called independently for the paired cfDNA and gDNA samples, using the methods disclosed above. In such embodiments, in addition to reporting the variant allele frequency, in both number of reads and aggregated plausibility among read groups, for the gDNA for all variants detected in the cfDNA, the variant allele frequency is reported, again in both number of reads and aggregated plausibility among read groups, for the cfDNA for all variants detected in the gDNA. The comparison of support metrics obtained for SNV and indels 1113 or fusion and rearrangements 1114 with the cleaned reads from gDNA 1108 and the associated read group information 1108 can be used to 1115 classify variants as putative tumor, putative germline or putative clonal hematopoiesis.

In some embodiments, variants are flagged as putatively tumor specific if the variant allele fraction in the cfDNA, measured either in reads or in aggregated plausibility among read groups, is at least 0.1 above the variant allele fraction in the gDNA. In some embodiments, variants are flagged as putatively tumor specific if the variant allele fraction in the cfDNA, measured either in reads or in aggregated plausibility among read groups, is at least 0.2 above the variant allele fraction in the gDNA. In some embodiments, variants are flagged as putatively tumor specific if the variant allele fraction in the cfDNA, measured either in reads or in aggregated plausibility among read groups, is at least 1.5 times higher than the variant allele fraction in the gDNA. In some embodiments, variants are flagged as putatively tumor specific if the variant allele fraction in the cfDNA, measured either in reads or in aggregated plausibility among read groups, is at least 2 times higher than the variant allele fraction in the gDNA. In some embodiments, variants are flagged as putatively tumor specific if the variant allele fraction in the cfDNA, measured either in reads or in aggregated plausibility among read groups, is at least 3 times higher than the variant allele fraction in the gDNA. In some embodiments, variants are flagged as putatively tumor specific if the variant allele fraction in the cfDNA, measured either in reads or in aggregated plausibility among read groups, is statistically different that measured in the paired gDNA sample, assuming a normal distribution, a binomial distribution, a Poisson distribution, or the like. In some embodiments, variants are flagged as putatively tumor specific if they match a criterion such as those listed above, but not if their variant allele fraction in the gDNA, measured either in reads or in aggregated plausibility among read groups is above a given threshold, which might 0.4, 0.5, 0.6, or another value.

In some embodiments, variants not flagged as putatively tumor specific are flagged as putatively germline if the variant allele fraction in the gDNA, measured either in reads or in aggregated plausibility among read groups, is comprised between 0.45 and 0.55 or is above 0.95. In some embodiments, variants not flagged as putatively tumor specific are flagged as putatively germline if the variant allele fraction in the gDNA, measured either in reads or in aggregated plausibility among read groups, is comprised between 0.40 and 0.60 or is above 0.90. In some embodiments, variants not flagged as putatively tumor specific are flagged as putatively germline if the variant allele fraction in the gDNA, measured either in reads or in aggregated plausibility among read groups, is statistically different from the expected 0.5 or 1.0 frequencies assuming a normal distribution, a binomial distribution, a Poisson distribution, or the like. In some embodiments, variants are first flagged as germline variants if they match one criterion, such as those listed here, and only those not listed as putatively of germline origin are evaluated for a possible tumor specificity.

In some embodiments, variants not flagged as putatively tumor specific nor as putatively germline are flagged as putatively stemming from clonal hematopoiesis. In some embodiments, thresholds are used to determine whether variants may stem from clonal hematopoiesis. In some embodiments, variants not fulfilling the criteria to be classified as putatively tumor specific, putatively germline, nor putatively clonal hematopoiesis are left unclassified.

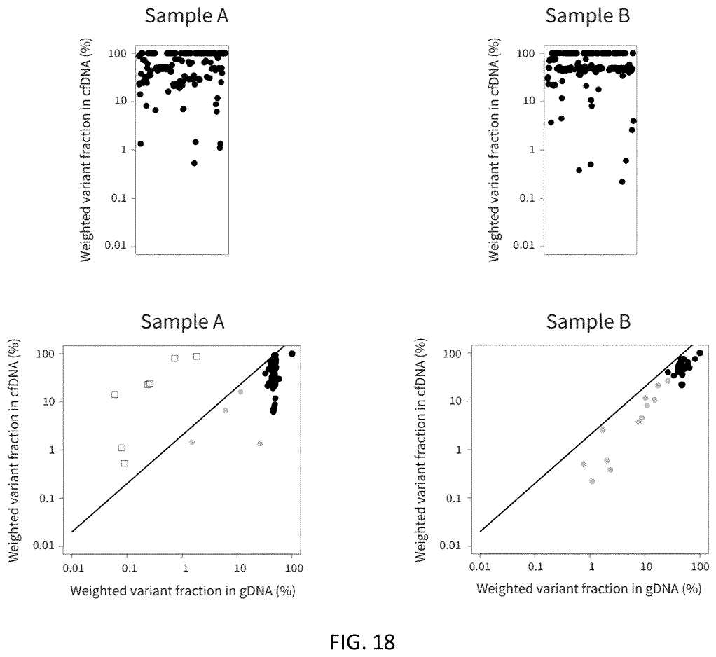

An example of the method is provided in FIG. 18. Batches of samples were prepared for sequencing using the method disclosed here. For each sample, 25 ng of cfDNA and 50 ng of paired gDNA were used to prepare a library. The ligated adapters contained a variable length spacer, with length variants obtained by truncating from a starting 9-bp sequence, so that each end of each molecule could receive any of ten different variable length spacers. After the whole-genome sequencing, the paired cfDNA and gDNA samples were pooled, each time combining 800 ng of the cfDNA library with 120 ng of the paired gDNA library. The probes used for hybrid capture targeted about 400,000 bp of genomic regions potentially relevant for cancers. A total of 13 PCR cycles were used post-capture, before cleaning to produce the capture library. The pools of libraries, containing 24 pairs of cfDNA-gDNA samples, were sequenced on one lane of Illumina NovaSeq S4, to reach an estimate of 100 million pairs of reads per sample.

Reads corresponding to different samples or to cfDNA versus gDNA from the same sample were demultiplexed based on unique dual indexes. For each read, the variable length spacer was identified based on the terminal sequence, and the length of the two variable spacers from reads of the same pairs were recorded. Variable length spacers and other adapter sequences were trimmed. Bad quality reads were discarded, and cleaned-trimmed reads were aligned to the human reference genome. Reads were assigned as potentially stemming from the same original molecule (read group) if they shared the same two variable spacer lengths and the same start and end positions with respect to the reference genome. For each position, metrics including the weighted number of read groups supporting the variant, weighted variant fraction, weighted read coverage, and weighted number of duplexes supporting the variants were calculated using methods disclosed herein. The background noise was calculated based on previously sequenced reference samples and the sequencing noise was calculated based on adjacent positions within the same sequencing data. Variants were called in the cfDNA samples if the support metrics exceeded pre-defined criterion and they were above those expected based on the background noise. For each variant called in the cfDNA sample, the weighted variant fraction in the paired gDNA sample was calculated using the same method.

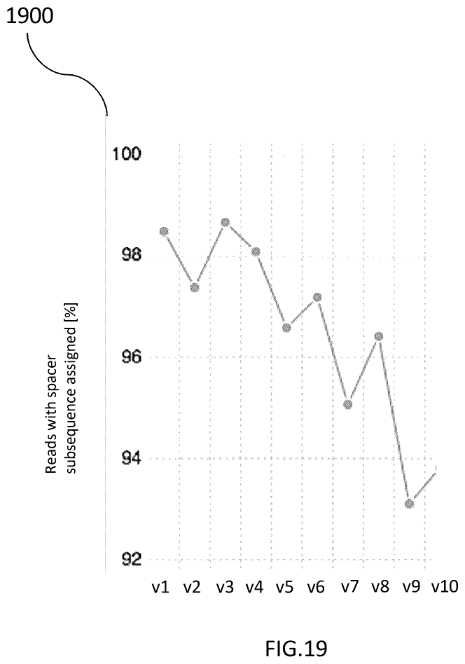

As illustrated in FIG. 18, on the top, the weighted variant fractions calculated based on the cfDNA show clusters of points at values around 50% and 100% in both samples, as expected for germline variants. In the absence of additional information, these clusters might however also contain tumor-specific variants. In both samples, variants are observed at low weighted variant fractions. While these values are not expected for germline variants, they might be compatible with either tumor variants or clonal hematopoiesis variants. FIG. 19 shows at the bottom the weighted variant fractions calculated from cfDNA and gDNA from the same sample. A majority of the variants with a high weighted variant fraction in the cfDNA also have a high weighted variant fraction in the gDNA, supporting their germline origin. In both samples, some variants with a low weighted variant fraction in the cfDNA have similar weighted variant fractions in the paired gDNA, supporting them as representing clonal hematopoiesis. In sample A, some variants with a high weighted variant fraction in the cfDNA however have a low weighted variant fraction in the gDNA, indicating they are likely of tumor origin. Such variants are absent from sample B. Importantly, the differences in tumor variants between samples A and B could not have been detected without paired cfDNA and gDNA samples.

In this example, variants were labelled as tumor specific if their weighted variant fraction in the cfDNA sample was at least twice higher than that in the paired gDNA samples (open squares). Other variants were labelled as germline if their weighted variant fraction in the gDNA sample exceeds 25% (black circles), and else as of likely clonal hematopoiesis origin (grey circles).

### Detection of Genomic Signatures

In some embodiments, the sequencing reads are used to infer genomic signatures.

In some embodiments, the inferred genomic signatures may include microsatellite instability, which may be inferred using methods known in the art, such as the method described in WO2021/156486.

In some embodiments, the inferred genomic signatures may include a genomic instability status. The genomic instability status may be inferred using methods known in the art, such as the method described in WO2022/023381. In embodiments where part of the whole-genome sequencing was mixed with the hybrid capture library prior to sequencing, the genomic instability index might be inferred considering only the reads from outside the regions targeted by the hybrid capture.

In some embodiments, the inferred genomic signatures may include a tumor mutational burden. The tumor mutational burden may be inferred using methods known in the art, such as the method described in EP4207204. The mutational mutation burden maybe be inferred from SNVs and indels detecting using the method disclosed here.

### Annotation and Reporting

In some embodiments, properties of the inferred single-nucleotide variants (SNV) and short insertion-deletions (indels), such as their positions with respect to coding sequences, their consequences for the encoded proteins, and their frequency in populations, may be computed or retrieved from existing databases. The likely pathogenicity of the variants may further be assessed using a combination of machine-learning tools and cross-referencing to in-house or clinical databases.

Clinically relevant information may be associated to the identified variants and biomarkers, including CNVs and fusions, by cross-referencing with knowledge databases. The information may include the likely responsiveness to different therapy options, available clinical trials, or scientific evidence.

The results of the analyses may be made available to the user via a user interface. In some embodiments, the user interface may be part of a stand-alone software. In some embodiments, the user interface may be part of a genomic platform, such as SOPHIA DDM™.

In some embodiments, the user interface may allow users to visualize the detected SNV, indel and fusions variants, together with the support values, in number of reads and/or in other support metrics generated using the invention disclosed. In embodiments where paired cfDNA and gDNA are analyzed jointly, the information may be available for both the cfDNA and gDNA samples.

In some embodiments, the user interface may further provide access to the inferred copy-number variants (CNV). In such embodiments, the estimated copy numbers may be reported for the targeted genomic regions, corresponding to chromosomal segments, genes, groups of exons, exons, or introns. For each region, significant deviation from the expected number of copies may be highlighted to the user. In some embodiments, equivocal deviations from the expected number of gene copies may be reported separately. In some embodiments, information about the inferred CNV, such as their previous observation in databases, their clinical associations, or their predicted functional significance, may be further reported to the user.

In some embodiments, the user interface further provides access to the inferred genomic signatures. In such embodiments, the genomic signatures might be reported with statistical support and confidence intervals.

In some embodiments, the user interface may be used to generate an automatically populated report. The report may be exported in a printable format, such as a Word document or a pdf. In some embodiments, the automatic report might be transferrable to other systems, such as those managing electronic health records.

In some embodiments, the user interface may allow the user to generate a customized report. In such embodiments, the user may be able to select the genes, variants, types of markers, and type of annotation to include in the report. The user may further be able to add custom notes and patient-specific information. The user may then be able to export the customized report in a printable format, such as Word document or a pdf documents. In some embodiments, the customized report might be transferrable to other systems, such as those managing electronic health records.

### EXEMPLARY EXPERIMENT

### Experiment 1

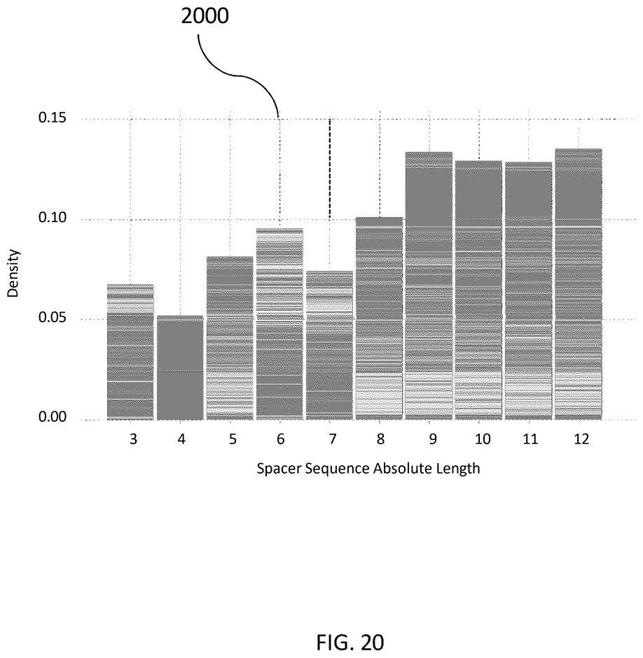

In a first experiment, we checked that all the proposed adaptors comprising a variable length spacer sequence as illustrated for instance in FIG. 7B can be ligated to DNA fragments to generate a library of DNA-adaptor products. As illustrated by the measurement 2000 of FIG. 20, when using a reaction mixture with the proposed adaptors during the ligation reaction, all spacer sequences adaptors can be ligated to DNA fragments and are almost equally represented in the final DNA library.

### Experiment 2

In a second experiment, we checked that the library of DNA-adaptor products as generated with the first experiment can be sequenced on an NGS platform such as an Illumina NextSeq sequencer and decoded by a genomic data analyzer 350 such as the Sophia Genetics Data Driven Medicine (Sophia DDM) bioinformatics platform. Each spacer sequence can be decoded from the raw FASTQ files out of the sequencer by the SOPHIA GENETICS Data Driven Medicine genomic data analyzer 350. The reads obtained show the expected sequence starting with the truncated spacer subsequence ending with the constant termination subsequence TS. FIG. 19 depicts a graph 1900 that shows that even for the longest length adaptors (which are more prone to base calling errors) more than 93% of the reads can be assigned to the expected spacer sequence by the bioinformatics workflow. In average, it is possible to properly identify the spacer sequence (and thus measure its variable length to form the numerical code tag) for around 95% of the reads.

### Experiment 3

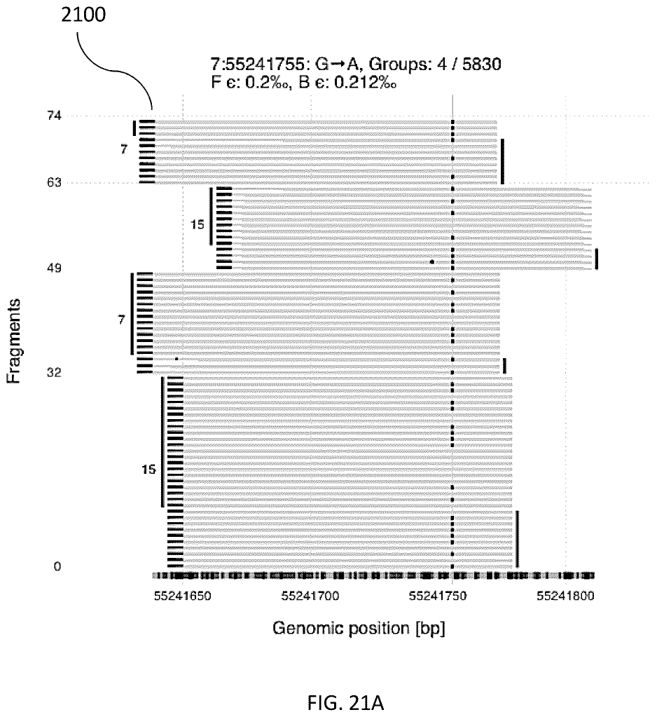

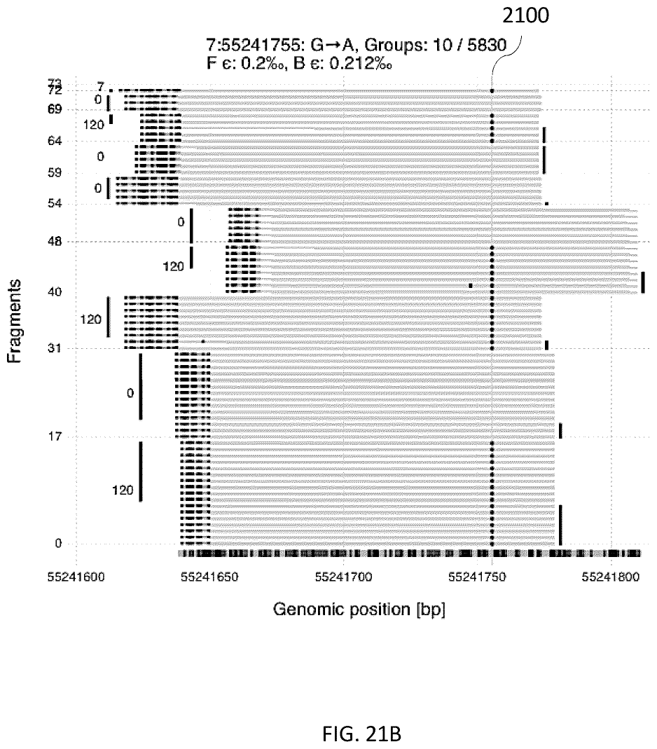

In a third experiment, using the raw reads as sequenced in the second experiment, we have compared with the IGV viewer their alignment results for a genomic analysis bioinformatics workflow (SOPHIA GENETICS DDM v5) ignoring the numerical code tagging, that is, grouping the reads obtained for a specific genomic position solely based on their start and end positions out of the alignment (FIG. 21A), versus the same genomic analysis bioinformatics workflow further adapted to group the reads obtained for a specific genomic position based on their start and end positions out of the alignment as well as the additional fragment tagging information of the proposed method numerical code, made of the measured variable adaptor spacer sequence lengths on both ends of the fragments (FIG. 21B).

As can be seen on FIG. 21A and FIG. 21B, the IGV viewer highlights the genomic position 2100 of a heterozygous SNPs. In a group of PCR duplicates without discriminating the origin fragment, in theory all the reads should either display the SNP (and the downstream variant caller 353 should measure Variant Fraction=1) or not display it (and the downstream variant caller 353 should measure Variant Fraction=0). In our practical experiment, however, as can be seen on FIG. 21A, only grouping the reads by their start and end information does not allow to accurately identify PCR duplicate groups as the actual variant fraction of the SNP differs from 0 or 1. This indicates that these groups contain DNA fragments deriving from at least two original DNA fragments. These original DNA fragments differed at the position of the SNP but were grouped together as they shared identical start and end positions. In contrast, as can be seen on FIG. 21B, adding the proposed numerical code as a tag allows to resolve these collisions by further subdividing and clustering the read groups of PCR duplicates having the same start and end positions into subsets originating from the same parent fragment according to their numerical code in the BAM file. In these subgroups, the Variant Fraction of the SNP can then be measured by the downstream variant caller 153 as either 0 or 1 as expected, thus demonstrating that the proposed numerical code in combination with the start and end position of the reads allows to discriminate PCR duplicates from colliding molecules.

### Experiment 4

Motivation: As will be apparent to those skilled in the art, calling variants at low variant allele fractions (VAF) is limited by sequencing errors and library preparation artefacts. A strategy to improve the analytical performance of NGS assays consists in exploiting the information provided by PCR duplicates for calling variants. Prior art solutions attempt to accurately identify PCR duplicate groups, for instance by mapping positions to identify PCR duplicates. However, the diversity of the shear points (and thus mapping positions) may not be sufficient to distinguish all original DNA molecules. Exogenous molecular barcodes have thus been introduced in order to provide additional information for the identification of PCR duplicate groups. However, there is no consensus today in the best industrial approach to generate such exogeneous barcodes, and a number of prior art solutions require the use of expensive library generation solutions, most of which have been primarily designed for use in a consensus sequencing workflow without benefiting from the most recent advances of probabilistic variant calling solutions. In contrast, the proposed variable length DNA-adaptors constructs aim at jointly facilitating both the exogenous identification of fragments and their efficient probabilistic genomic analysis to further improve the sensitivity and specificity of low frequency variant detection. This is demonstrated by a dedicated experiment, as will now be detailed.

Sample Preparation: The nucleosomal DNA of six cell lines was spiked-in the nucleosomal DNA of a seventh cell line in different ratios in order to generate three samples with a series of single nucleotide variations (SNVs) at the following variant allele frequencies: 0.5-4%, 0.25-2% and 0.1-0.8%.

Targeted Library Preparation: Whole-genome libraries were prepared in duplicate from 25 ng of each DNA mix using SOPHIA GENETICS library preparation kit following manufacturers instructions with minor modifications. Briefly, after end-repair and A-tailing, the DNA fragments of each sample were ligated either to standard, nonbarcoded, adaptors or to a set of variable length adaptors comprising a variable length spacer sequence) (LT5=3, L₅=9, so as to produce 10 different DNA adaptors of respective lengths 3 to 12 nucleotides). The libraries were then amplified using indexed, Illumina-compatible primers. Whole-genome libraries were captured using SOPHIA GENETICS capture protocol and a SOPHIA GENETICS catalog panel (footprint: 56 Kb) covering 23 of the SNVs present in the DNA mixes.

Data Analysis: The libraries from the variable length adaptors construction experiment were first pre-processed. The position of the constant subsequence at the beginning of the forward and reverse reads was determined. Then, the length of the adaptors present on both sides of each DNA fragment was used to generate a combinatorial code that was added to the read header prior to trimming the variable length adaptor sequences. Then, the reads of all libraries were aligned to the genome using the BWA-MEM aligner. Groups of PCR duplicates were identified using the fragment mapping position and the aforementioned combinatorial code. Variant calling was performed either by probabilistic sequencing or duplex consensus sequencing. For probabilistic sequencing, the posterior probability of the group of PCR duplicates being issued from a molecule carrying a SNV was computed and used to assign a quality score to each identified PCR duplicate group.

### Results:

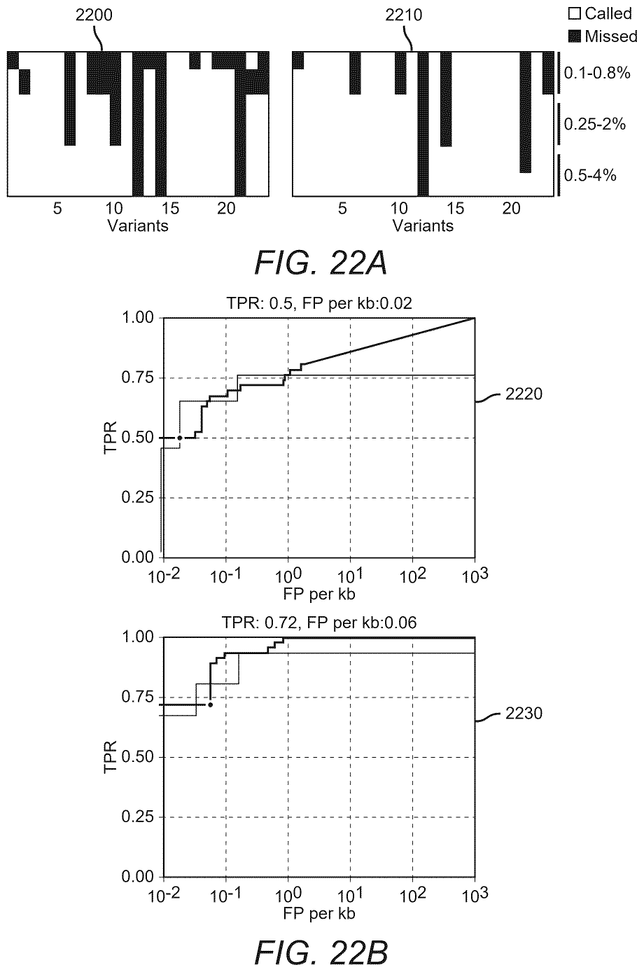

FIGS. 22A-22B show that the proposed variable length adaptors facilitate the detection of rate variants in artificial nucleosomal DNAs. FIG. 22A shows the variant calling results 2200 for 3 samples (25 ng DNA input) analyzed in replicates and harboring 23 SNVs at 3 distinct VAFs ranges (sample 1:0.5-4%; sample 2:0.25-2%; and sample 3:0.1-0.8%) when prior art standard adaptors are used. FIG. 22A further shows the variant calling results 2210 obtained when the proposed variable length adaptors are used. Out of the 144 SNVs tested in this experiment, only 107 were detected when using standard adaptors. Using SLA libraries, the sensitivity improved with 123 variants being called. FIG. 22B further compares the ROC curve 2220 showing the performance of variant calling in terms of true positive rate (TPR) versus false positive rate (FP) when respectively using probabilistic sequencing (dark grey) or duplex consensus sequencing (light grey) in the samples harboring variants with VAF ranging between 0.1-0.8% and processed using the prior art standard adaptors. FIG. 22B further compares the ROC curve 2230 showing the performance of variant calling in terms of true positive rate (TPR) versus false positive rate (FP) when respectively using probabilistic sequencing (dark grey) or duplex consensus sequencing (light grey) in the samples harboring variants with VAF ranging between 0.1-0.8% and processed using the proposed variable length adaptors.

### Advantages of the Proposed Method:

The proposed method thus facilitates the NGS bioinformatics identification of variants even out of low input DNA amounts while only requiring the ligation of a few predefined variable length adaptors to produce a library of DNA-adaptor products suitable for various downstream NGS workflows. As will be apparent to those skilled in the art of high-throughput sequencing data processing, in the genomic analysis workflow the trimming of the adaptor sequence during read pre-processing need to be accurate, since over-trimming will lead to a loss of sequencing coverage and under-trimming can introduce sequencing artefacts. Prior art variable length adaptors that do not possess a constant termination subsequence signal (TS) do not allow to identify the boundary between the barcode and the beginning of the insert DNA fragment. As a result, they usually require trimming on the adaptor full length Lmax, and cause reduced coverage.

Moreover, the synthesis of adaptors being expensive, in a routine clinical practice workflow it is preferable use as many barcodes as required to resolve collisions for a specific application. When using a limited number of barcodes, it is important that those are uniformly represented in the final library, otherwise the effective number of barcode combination is reduced and may not suffice anymore. Having a constant termination subsequence TS at the extremity of each barcode prevents ligation sequence-specific biases and allows thus to have a more uniform barcode usage.

Furthermore, depending on the actual sequencing technology, for instance with Illumina sequencers, having base imbalance in the first sequencing cycles can reduce the sequencing quality. This can become an issue when using a limited number of random barcodes. Using predetermined sets of spacer sequences of variable length, that may be designed such as to have a balance base composition at each sequencing cycle, allows to maintain a high sequencing quality.

What is claimed is:

1. A method for detecting genetic variants in a sample, the method comprising the steps of:
a. preparing a nucleic acid for sequencing using a high-throughput sequencing platform,
wherein the preparing comprises a least one PCR amplification reaction,
wherein the preparing comprises, before the first PCR amplification reaction, incorporating molecular identifiers that can differ among molecules;
b. subjecting the preparation to high-throughput sequencing;
c. processing sequencing reads to remove adapter sequences and bad quality sequence;
d. aligning cleaned sequencing reads to a reference genome;
e. for each position in genomic regions of interest, assessing the likelihood of a genetic variant being present in the sample based on at least one aggregated plausibility metric that considers the distribution of signal among groups of reads with distinct molecular identifiers that overlap with the position.

2. The method of claim 1, wherein the molecular identifiers are provided by the start and end sites of the nucleic acid fragment compared to a reference genome.

3. The method of claim 2, wherein the molecular identifiers further include variable length spacers incorporated as part of adapters,
wherein the variable length spacers include a variable subsequence that can differ among adapters and a constant termination sequence that is used to identify the end of the variable length spacer in the sequencing reads.

4. The method of claim 1, wherein the plausibility is calculated for each group of reads sharing the same molecular identifiers and then aggregated among groups of reads with distinct molecular identifiers overlapping the genomic position.

5. The method of claim 1, wherein the plausibility is calculated for each possible state, wherein the possible states represent any of distinct nucleotides, insertions of nucleotides compared to the reference genome, deletions of nucleotides compared to the reference genome, or groups of nucleotides.

6. The method of claim 5, wherein inferring the plausibility is based on at least one of the number of reads supporting a given state, the quality of the reads supporting a given state, the quality of the positions supporting a given state, or the quality of positions adjacent to the positions supporting a given state.

7. The method of claim 6, wherein the inferred plausibility is decreased if the level of support for the state differs among forward and reverse reads or among reads originating from each of the two strands in double-stranded starting molecules.

8. The method of claim 5, wherein calculating the plausibility for a given state involves comparing the support for the most highly supported and the support for the second most highly supported within each group of reads sharing the same molecular identifiers.

9. The method of claim 1, wherein assessing the likelihood of a genetic variant being present at a given genomic position in the sample involves calculating a weighted support metric based on at least one plausibility metric aggregated among groups of reads sharing the same molecular identifiers that overlap with the genomic position.

10. The method of claim 9, wherein the weighted support metric represents the plausibility summed across groups of reads sharing the same molecular identifiers that support the same state at a given position, the fraction of the aggregated plausibility for a given state out of the aggregated plausibility for all states, the plausibility summed across groups of reads sharing the same molecular identifiers for which both forward and reverse reads support the same state at a given position, or the plausibility summed across groups of reads sharing the same molecular identifiers for which reads originating from both strands in the original double-stranded nucleic acid support the same state at a given position.

11. The method of claim 9, wherein the aggregated plausibility includes a correction factor to penalize some states.

12. The method of claim 9, wherein the aggregated plausibility includes at least one correction factor based on at least one of the concordance between forward and reverse reads, the concordance between the reads originating from each of the two strands in the original molecule, or the amount of support expected at the position in the absence of a variant in the original sample.

13. The method of claim 1, wherein assessing the likelihood of a genetic variant being present at a given genomic position in the sample involves comparing an aggregated plausibility metric to a pre-defined threshold.

14. The method of claim 1, wherein assessing the likelihood of a genetic variant being present at a given genomic position in the sample involves comparing an aggregated plausibility metric to the value of the aggregated plausibility metric expected in the absence of a genetic variant.

15. The method of claim 1, wherein assessing the likelihood of a genetic variant being present at a given genomic position in the sample is based on multiple metrics, at least one of which is a plausibility metric aggregated across groups of reads with distinct molecular identifiers.

16. The method of claim 1, wherein the starting nucleic acid is DNA, in the form of genomic DNA, cell-free DNA, or complementary DNA obtained via reverse transcription of RNA of cell-free DNA.

17. The method of claim 16, wherein cell-free DNA and genomic DNA of a same subject are analyzed in parallel, wherein the support metric for each genetic variant are compared between the cell-free DNA and the paired genomic DNA to distinguish putatively somatic from putatively germline variants.

18. The method of claim 17, wherein the subject suffers to or is suspected to suffer from a cancer, and wherein the comparison of the support metric for each genetic variant between the cell-free DNA and the paired genomic DNA is used to distinguish putative germline variants, putative clonal hematopoiesis variants, and putative tumor variants.

19. The method of claim 1, wherein the genetic variants with a probability above a given threshold are reported together with support values to a user.
